SEQUENCE LISTING (4 sequences)

SEQ ID NO: 1 - protein, 4 aa, Artificial sequence
  Chemically synthesized cAMP substrate
AFPF

SEQ ID NO: 2 - protein, 5 aa, Artificial sequence
  Chemically synthesized cAMP substrate
AAEPR

SEQ ID NO: 3 - protein, 6 aa, Artificial sequence
  Chemically synthesized cAMP substrate
CFPACF

SEQ ID NO: 4 - protein, 16 aa, Artificial sequence
  Calmodulin sequence motif
KHAAQRSTETALYRKM

(19) United States
(12) Patent Application Publication
(10) Pub. No.: US 2007/0244165 A1
(43) Pub. Date: Oct. 18, 2007

(54) Method for Identifying and Producing Effectors of Calmodulin-Dependent Peptidyl-Prolyl Cis/Trans Isomers

(75) Inventors: Gunter Fischer, Halle (DE); Frank Edlich, Halle (DE); Matthias Weiwad, Halle (DE); Franziska Jarczowski, Roblingen (DE); Gerhard Kullertz, Halle (DE)

Correspondence Address: KAGAN BINDER, PLLC, SUITE 200, MAPLE ISLAND BUILDING, 221 MAIN STREET NORTH, STILLWATER, MN, 55082, US

(73) Assignee: MAX-PLANK-GESELLSCHAFT ZUR FORDERUNG, Berlin (DE)

(21) Appl. No.: 10/587,326

(22) PCT Filed: Jan. 24, 2005
(86) PCT No.: PCT/EP05/00656

§ 371 Date: Jun. 4, 2007

(30) Foreign Application Priority Data
Jan. 22, 2004 (DE) 10 2004 003 362.5

(51) Int. Cl. A61K 31/44; C07D 211/72; C12Q 1/533

(52) U.S. Cl. 514/348; 435/7.4; 546/296

(57) ABSTRACT

The present invention relates to a method for identifying and producing effectors of peptidyl-prolyl cis/trans isomerases which can be activated by calmodulin. The invention also relates to the use of the identified effectors for the production of medicaments and to screening methods and kits.

The present invention relates to a method for the identification and production of effectors of peptidyl-prolyl cis/trans isomerases that are susceptible to activation through calmodulin. The invention further relates to the use of the identified effectors for the production of medicaments, screening methods and kits.

Peptidyl-prolyl cis/trans isomerases, in the following referred to as PPIases, are classified under the EC No. 5.2.1.8 in accordance with the recommendations of the “Nomenclature Committee of the International Union of Biochemistry and Molecular Biology” (“Enzyme Nomenclature”, Academic Press, 1922). The main representatives of this enzyme class were discovered and defined on the basis of their enzymatic activity towards appropriate substrates, e.g. cyclophilin (Fischer et al., Nature. 337 (1989):476-8); FKBP12 (Harding et al., Nature. 341(1989):758-60) or parvulin (Rahfeld et al., FEBS Letters. 352(1994):180-4). PPIases are able to catalyse the cis/trans isomerization of prolyl peptide bonds in oligopeptides and proteins. At an international level, proteins that have not been classified so far are often classified under this enzyme class by means of primary sequence comparison using permanently updated databases such as Swiss-Prot, TrEMBL (e.g. Nucleic Acid Res. 31:365-370 (2003) or CELERA (cds@celera.com) as well as appropriate comparison algorithms (such as Bioinformatics 15:219-277 (1999) or U.S. Pat. No. 6,023,659). The quality of this classification is assessed using appropriate score values.

Another possibility of assigning unknown proteins to the class of PPIases consists in determining the PPIase activity by means of appropriate PPIase activity assays such as isomer-specific proteolysis (Fischer et al., Biochim. Biophys Aacta 43(1984), 1101; Fischer et al., Nature. 337(1989): 476-8), magnetic nuclear resonance spectroscopy (Kern et al., Biochemistry 34(12995)13598, Reimer et al., Biochemical J. 326(1997)181) or by using other spectrographic determination methods known to the person skilled in the art (e.g.: Janowski et al., Analytical Biochemistry 252(1997), 299; Garcia-Echeverria et al., Biochem. Biophys. Res. Commun. 191(1992), 2758). Despite the use of the various methods for the detection PPIase activity mentioned above, it turned out that some enzymes assigned to PPIases, such as FKBP38 (Shirane Nature Cell Biology 5(2003)1) exhibit only a low or scarcely detectable PPIase activity towards typical PPIase substrates in comparison to known PPIase representatives such as cyclophilin, FKBP12 or parvulin. Typical PPIase substrates have a peptidyl prolyl peptide bond, they are known to the person skilled in the art and are described in numerous passages of the technical literature accessible to those skilled in the art, e.g.: Clinical Chemistry. 44(3):502-8, 1998; Analytical Biochemistry. 252(2): 299-307; 1997; Biochemistry. 34(41):13594-602, 1995; Biochemistry. 30(25):6127-34, 1991; Biochemistry. 30(25): 6127-34, 1991; Journal of Molecular Biology. 271(5): 827-37, 1997, or are mentioned in patent specifications, as for instance in CA2334812; EP0647713; WO0142245; EP0360029.

Furthermore, since 1986, protein complexes have been described that consist of a molecule of a PPIase, a low-molecular agent such as cyclosporin A or FK506, the protein phosphatase calcineurin, calmodulin and of up to 4 calcium ions: e.g.: Cell Biochemistry & Biophysics. (30(1):115-51, 1999; Bioorganic & Medicinal Chemistry. 5(2):217-32, 1997; Current Opinion in Structural Biology. 6(6):770-5, 1996; FASEB Journal. 9(1): 63-72, 1995. Transplantation Proceedings. 18(6 Suppl. 5):219-37, 1986; Science. 233(4767):987-9, 1986. In none of these cases, it was possible to provide evidence of an activation of the PPIase activity of these complexes. Some of the studies even indicate an inhibition of the PPIase activity in this complex.

Calmodulin is a common intracellular Ca²⁺ receptor present in animal and plant cells that mediates numerous Ca²⁺-regulated processes. An increase in the cytoplasmatic calcium level caused by the opening of calcium channels in the plasma membrane or a membrane of intracellular storage vesicles leads to an activation of calmodulin. Many enzymes, pumps, membrane transporter proteins and other target proteins are regulated by Ca²⁺/calmodulin, the majority of effects being induced not directly by calmodulin but via Ca²⁺/calmodulin-dependent protein kinases. In some cases calmodulin is a separate regulatory subunit of an allosteric enzyme (e.g. phosphorylase kinase). Apparently, calmodulin identifies the different target proteins by positively charged amphipathic alpha-helices as both Ca²⁺ calmodulin lobes have hydrophobic sequence regions, which are surrounded by negatively charged regions and, thus, exhibit complementarity to positively charged amphiphilic alpha-helices (P. Cohen and C. B. Klee (eds.) Calmodulin. Elsevier, Amsterdam, 1988). Although parts of the primary sequence seem to be conserved, there are significant differences between the individual species, for example between the human protein and the yeast protein.

As is generally known, the interaction of calmodulin with enzymes does not necessarily lead to an increase in their activity. Examples have been described in which the enzyme activity is either not at all affected, e.g. Biochemical Journal. 368 (Part 1):145-157, 2002; Biochemical Journal 365 (Part 3):659-667, 2002 or an inhibitory effect was observed (e.g.: Biochemistry. 42(9):2740-2747, 2003; National Academy of Sciences of the United States of America. 99(12):8424-8429, 2002; Biochemistry. 40(41):12430-12435, 2001; Proceedings of the National Academy of Sciences of the United States of America. 98(6):3168-3173, 2001).

Furthermore, since 1992, various groups have been able either to predict or to directly detect a direct interaction of PPIases with calmodulin, independent of the “multimer complexes” described above, though without providing proof of an activation of the PPIase activity by calmodulin. Cf.: Plant Molecular Biology. 48(4):369-381, 2002; Mini-Reviews in Medicinal Chemistry. 1(4):377-97, 2001; Planta. 215(1):119-26, 2002; Plant Molecular Biology. 48(4):369-81, 2002, Journal of Cellular Biochemistry. 84(3):460-71, 2002; Journal of Biological Chemistry. 276(42):38762-73, 2001; Trends in Plant Science. 6(9):426-31, 2001; Structure. 9(5):431-8, 2001; Developmental Biology. 225(1):101-11, 2000; Plant Physiology. 119(2):693-704, 1999; Planta. 205(1):121-31, 1998; Journal of Biological Chemistry. 272(51):32463-71, 1997; Plant Molecular Biology. 32(3):493-504, 1996; Molecular & General Genetics. 252(5):510-7, 1996; Biochemical & Biophysical Research Communications. 209(1):117-25, 1995; Journal of Biological Chemistry. 268(18):13187-92, 1993; Proceedings of the National Academy of Sciences of the United States of America. 89(22):10974-8, 1992; Proceedings of the National Academy of Sciences of the United States of America. 89(14):6270-4, 1992. In none of these studies, however, the activation of one of the PPIases activities examined through the addition of calmodulin could be described. Effectors of PPIases known up to date, such as cyclosporin A (e.g. U.S. Pat. No. 4,722,999), FK506 (e.g. U.S. Pat. No. 5,457,182) or rapamycin (e.g. U.S. Pat. No. 4,885,171), are considered highly efficient medicaments for human use.

The problem underlying the present invention consisted in providing means for a method for the identification and/or the production of an effector of a calmodulin-dependent peptidyl-prolyl cis/trans isomerase. The solution of this technical problem is to provide medicaments exhibiting a specific activity.

The technical problem is solved by providing, in accordance with the invention, the embodiments characterised in the patent claims and is based on the unexpected teaching, which is contrary to the teaching of the state of the art, that, under specific circumstances, PPIase activity of calmodulin binding PPIases can be activated by calmodulin.

Thus, the present invention relates to a method for the identification and/or the production of an effector of a calmodulin-dependent peptidyl-prolyl cis/trans isomerase (CaMAP) consisting of the following steps:
(a) mixing of appropriate amounts of a CaMAP or a CaMAP peptide fragment/derivative with an appropriate amount of calmodulin or of a calmodulin fragment/derivative in an appropriate reaction solution with and without the effector;
(b) adding an appropriate amount of an appropriate CaMAP substrate,
(c) measuring CaMAP activity; and
(d) detecting that the effector is
   (i) an inhibitor if the CaMAP activity in the reaction solution with the effector is lower than in the reaction solution without the effector; or
   (ii) an activator if the CaMAP activity in the reaction solution with the effector is higher than in the reaction solution without the effector.

In the context of the present invention, the term “effector” defines molecules that modulate the enzymatic activity of the peptidyl-prolyl cis/trans isomerases (modulators). In accordance with the invention, this definition comprises inhibitors and activators. “Inhibitors” are defined as molecules which inhibit a particular enzymatic activity. The term “activators” defines molecules which enhance a particular enzymatic activity. The effectors that can be analysed according to the present method are substances of all known classes suited for the method due to their physical-chemical properties. The analysable effectors comprise inorganic as well as organic molecules. The effectors can be chemically synthesised or can be obtained by isolation from natural sources, such as live organisms. Effectors are preferably peptides, polypeptides, sugar molecules, lipids or combinations of these molecule groups. More preferably, the analysable effectors are peptides or polypeptides or derivatives thereof. Especially preferred, the effectors comprise molecules that are known to interact with peptidyl-prolyl cis/trans isomerases such as peptide inhibitors, antibodies, lectines or fragments or derivatives thereof, the inhibiting or activating activity of which can be assayed by means of the present method of the invention.

“Calmodulin-dependent peptidyl-prolyl cis/trans isomerases (CaMAPs)” within the meaning of the invention can be obtained by various methods by the person skilled in the art, such as described in the following. In accordance with the invention, the term CaMAP also comprises “CaMAP peptide fragments/derivatives” designating CaMAPs that are truncated or modified by different methods known to the person skilled in the art and exhibiting peptidyl-prolyl cis/trans isomerase activity.

Motif search: Databases accessible to those skilled in the art, such as Swiss-Prot; TrEMBL; Trenew; Trest; Trgen; Trome etc., allow searching for PPIases having motifs that are typical for binding of calmodulin. Typical motifs are known to the person skilled in the art and have been described repeatedly, for instance in Journal of Biological Chemistry. 277 (2002) 14681; Journal of Biological Chemistry. 276(10):7129-35, 2001; Journal of Biological Chemistry. 273(18):10819-22, 1998; Journal of Biological Chemistry. 269(42):26431-7, 1994; Journal of Biological Chemistry. 276(10):7129-35, 2001; Plant Physiology. 123(4):1495-506, 2000; Journal of Biological Chemistry. 275(28):21121-9, 2000; FEBS Letters. 455(3):367-71, 1999; Chinese Journal of Biotechnology. 14(3): 165-71, 1998; FASEB J. 1997 April;11(5):331-40; Nature 410 (2001) 1120-1124.

In the following, Example 4 will illustrate a search strategy of the invention on the basis of the helical calmodulin motif KHAAQRSTETALYRKM which is used to find potential CaMAPs in the databases known to the person skilled in the art. The definite CaMAP classification is accomplished subsequently by means of one or more activity assays known to the person skilled in the art. Appropriate methods of detecting activity in accordance with the invention comprise, amongst others, the above-identified methods of isomer specific proteolysis, magnetic nuclear resonance spectroscopy, or other spectrographic determination methods known to the person skilled in the art. In this context, reference is also made to Examples 1-3 illustrating typical determination methods of PPIase activity.

Interaction with calmodulin: Other strategies of the invention used to detect CaMAPs in accordance with the invention are based on the binding affinity between calmodulin and binding protein. As already described, by adding appropriate amounts of calmodulin and under appropriate conditions, it was unexpectedly possible in the present process of the invention to increase the PPIase activity of several PPIases towards appropriate substrates by at least one order of magnitude, with optimum conditions allowing an increase by several orders of magnitude, e.g. as described in the Examples 1-3 a ten thousand fold increase. As shown in Example 1, CaMAP activity can be activated by calmodulin dose-dependently. Calmodulin from various animal species can be used to activate CaMAP. As described in Example 1, it can be calmodulin obtained from bovine brain or human calmodulin obtained by molecular-biological methods (Example 2).

There are numerous methods and procedures to detect the interaction comprising automated search strategies by means of devices that were developed for screening (e.g. Biosensor [Vaccine. 18(3-4):362-70, 1999; Journal of Biological Chemistry. 273(31):19691-8, 1998], calorimetry [Biochimica et Biophysica Acta. 386(1):155-67, 1975; Yao Hsueh Hsueh Pao—Acta Pharmaceutica Sinica. 35(10):774-7; 2000]; correlation spectrography [Current Opinion in Chemical Biology. 2(3):397-403, 1998; Proceedings of the National Academy of Sciences of the United States of America. 95(4):1421-6, 1998]). Thus, in accordance with the invention, the potential binding protein or the effective calmodulin is preferably bound to a macroscopic matrix, such as agarose (calmodulin agarose for affinity studies is available on the market; e.g. Sigma order no.: P4385), and subsequently freed from unbound substances by washing in order to subsequently identify the interaction partners by appropriate strategies, such as SDS-PAGE and mass spectrometry. The identification of calmodulin interaction partners was several times accomplished successfully in this way, cf. the following studies: Plant Physiology. 116(2):845-851, 1998; Plant Journal. 24(3):317-326, 2000; Molecular & Cellular Biochemistry. 183(1-2):183-191, 1998; Planta 205(1):121-131, 1998; Journal of Biological Chemistry. 273(2):677-680, 1998. Another possibility to detect interaction consists in stabilising the interaction by chemical modification, known “as crosslinking” to the person skilled in the art and described amongst others in the following studies: Biochemistry. 40(26):7903-7913, 2001; Biochemistry. 37(23):8378-8384, 1998; Biochemistry. 35(14):4375-4386, 1996; Journal of Biological Chemistry. 271(5):2651-2657, 1996. Using this technique, also AtFKBP42, which is responsible for a phenotype in Arabidopsis and is designated as tr:Q9LDC0 in the database of Example 4, was discovered (Plant Journal. 32(3):263-376, 2002).

Structure modification: A further strategy of the invention used to identify CaMAPs in accordance with the invention is based on the fact that the binding of calmodulin to CaMAP often modifies the molecular geometry of CaMAP in such a way that it can be measured by spectroscopic methods. Spectroscopic methods for detecting these structural changes are known to those skilled in the art. A typical structural modification is described in Example 11.

Activity modification: Preferably, the interaction of calmodulin and CaMAP can be identified on the basis of the PPIase activity increase which is caused by the interaction. Typical applications are summarised in Examples 1, 2, 3, 5 and 8 hereafter. In the present invention, this technique allowed identifying amongst others MzFKBP66 and AtFKBP42 as CaMAPs.

According to the present invention, PPIases activable by calmodulin may diverge from nucleotide sequences available in databases. Thus, it is possible to obtain a protein with new properties by systematic modifications via genetic engineering or chemical means or by direct chemical synthesis. Such modifications may aim at tailoring CaMAPs for specific roles in technology and medicine. This includes the production of CaMAPs with 1) higher stability with respect to heat, extreme pH-values, oxidative atmospheres and organic solvents; 2) enhanced or new substrate specificity; 3) modified properties that facilitate the recovery in follow-up procedures; 4) modified properties which cause a constitutive activity of CaMAP. As conformation and properties of proteins are determined by their primary sequence, the focus is essentially on the systematic modification of the amino acid sequences of existing proteins. Alternatively, small proteins can be synthesised chemically (peptide synthesis). Chemical synthesis allows further regulation of the secondary structure of the protein since unnatural amino acids can be introduced, such as 2,2-dimethylglycine the conformation of which is restricted and, thus, allows a stable secondary structure. It is also possible to chemically modify native enzymes with the aim of maintaining the normal properties (for example enzyme activity) while greater stability is achieved. A method generally applied consists in synthesising genes that code for a sought-after polypeptide sequence. Hybrid genes can be synthesised chemically by adding segments of natural genes to chemically synthesised DNA sequences. Alternatively, new primary sequences can be formed by using synthetic DNA sequences to enlarge natural genes or to replace segments of natural genes. The synthetic DNA or the hybrid DNA formed is subsequently inserted into a plasmid in order to synthesise the intended protein.

Molecular-biological modification: The primary sequence of a CAMAP can be modified by methods known to the person skilled in the art in such a way that single or several amino acids of the primary sequence are replaced by others. Thus, to enhance the solubility of a CaMAP in Example 9, a glycine in position 2 is replaced by an arginine. The scope of the invention also comprises extending the primary sequence at the N- or C-terminal end using single amino acids, oligopeptides or whole proteins. The scope of the invention further comprises producing only partial peptide regions of the natural CaMAPs or of the CaMAPs modified by molecular-biologically methods. Thus, Example 9 describes the production of a CaMAP with a truncated N-terminal. CaMAP or CAMAP peptide fragments/derivatives that are genetically modified and are particularly appropriate for searching effectors of CaMAPs are recognisable by the activating effect of calmodulin of the invention on the PPIase activity of the CaMAP. In accordance with the invention, these activated CaMAPs and peptide fragments/derivatives, respectively, are preferred.

After identifying potential CaMAP sequences in databases, the protein can be prepared from the nucleotide sequence by accepted methods known to the person skilled in the art. cDNA clone databases and genome libraries provide DNA sources for cloning. In order to transfer and introduce foreign DNA sequences in a host cell DNA vectors are used into which the desired sequences are introduced. By using restriction endonucleases of different specificity, the DNA vectors and cellular genomes can be cleaved at chosen positions. If vector and donor DNA are cleaved with the same enzyme, the terminal ends are complementary and can be hybridised and subsequently covalently linked by a DNA ligase (polynucleotide ligase). Depending on the vector and host system, a great number of methods, known and available to those skilled in the art, have been developed to introduce the recombinant DNA that is produced by gene-manipulation into a host cell. Example 10 describes the molecular-biological production of a CaMAP.

Post-translational modifications: Apart from the modification of the primary sequence described above, CaMAPs may be modified post-translationally. Thus, CaMAPS can, for example, be acetylated, methylated, ubiquitinised or phosphorylated. Numerous post-translational modifications taking place in vivo can be accomplished by the person skilled in the art also in vitro. Thus, at least in vitro, CaMAP can be phosphorylated by using different protein-serine/threonine and protein-tyrosine kinases. Depending on the type of source and the isolation method, CaMAP exhibiting different post-translational modifications is obtained. Surveys of post-translational modifications of proteins are known to those skilled in the art and have been described in numerous publications, e.g.: Annals of the New York Academy of Sciences 663:48-62, 1992; Advances in Protein Chemistry. 37:247-334, 1985; Science. 198(4320):890-6, 1977; Current Opinion in Rheumatology. 14(3):244-9, 2002; Biomolecular Engineering. 18(5):213-20, 2001; International Journal of Biochemistry. 24(1):19-28, 1992). Example 10 illustrates the radioactive labeling of a CaMAP. CaMAPs and CaMAP peptide fragments/derivatives that are post-translationally modified and are particularly appropriate to search for effectors of CaMAPs are recognisable by the activating effect of calmodulin on the PPIase activity of CaMAP as set forth in the invention. In accordance with the invention, these activated CaMAPs and CaMAP peptide fragments/derivatives, respectively, are preferred.

Protein-chemical modification: According to invention, it can be an advantage to modify the CaMAP chemically. The necessary methods and procedures are known to those skilled in the art as protein-chemical methods. Besides the modification of lysines (Journal of Biological Chemistry. 273(43):28516-28523, 1998; Biochemica et Biophysica Acta. 844(2):265-9, 1985), the oxidation or carbethoxylation (Pharmacology. 26(5):249-57, 1983; Biochemistry. 17(19):3924-8, 1978), various chemical modifications are possible. Known techniques of chemical modification are described amongst others in: Current Opinion in Biotechnology. 10(4):324-30, 1999; Current Opinion in Chemical Biology. 5(6):696-704, 2001; Biochemistry-Russia. 63(3):334-44, 1998; Biotechnology & Applied Biochemistry. 26 (Pt 3):143-51, 1997; Biological Research. 29(1):127-40, 1996; Methods in Molecular Biology. 35:171-85, 1994; Methods in Molecular Biology. 32:311-20, 1994; Nature Cell Biology. 5(1):28-37, 2003). Example 10 illustrates radioactive labeling of a CaMAP. CaMAPs and CaMAP peptide fragments/derivatives that are chemically modified and are particularly appropriate to search for effectors of CaMAPs are recognisable by the activating effect of calmodulin on the PPIase activity of CaMAP as set forth in the invention. These activated CaMAPs and CaMAP peptide fragments/derivatives, respectively, are preferred in accordance with the invention.

According to the invention, calmodulin comprises all polypeptides of this molecule class known to the person skilled in the art that are suitable for activating CaMAP activity. In accordance with the invention, calmodulin fragments and/or calmodulin derivatives that, in the method of the invention, result in an activation of the peptidyl-prolyl cis/trans isomerase activity are as well included. The detection of the enhancement of the peptidyl-prolyl cis/trans isomerase activity can be accomplished by the above-described method that is known to those skilled in the art. Calmodulin in accordance with the invention can vary considerably. Thus, the systematic modification of a protein can be accomplished by genetic or chemical means or by direct chemical synthesis of a protein having new properties. The aim of such modifications consists in tailoring proteins for specific roles in technology and medicine. This encompasses the production of proteins having 1) higher stability with regard to heat, extreme pH-values, oxidative atmospheres and organic solvents; 2) enhanced or new substrate specificity and 3) modified properties facilitating the recovery in follow-up procedures. As conformation and properties of the proteins are determined by their primary sequence, the focus is essentially on the systematic modification of the amino acid sequences of existing proteins. Alternatively, small proteins can be synthesised chemically (peptide synthesis). Chemical synthesis allows further regulation of the secondary structure of the protein since unnatural amino acids can be introduced, such as 2,2-dimethylglycine the conformation of which is restricted and, thus, allows a stable secondary structure. It is also possible to chemically modify native enzymes with the aim of maintaining the normal properties (for example the enzyme activity) while greater stability is achieved. Thus, resistance against proteolytic degradation in the body was imparted to the tissue plasminogen activator that is used in thrombosis treatment by modifying the surface lysine residues through a reaction with an acid anhydride. A method generally applied consists in synthesising genes that code for a sought-after polypeptide sequence. It is possible to chemically synthesise synthetic genes from up to 100 nucleotides. Hybrid genes can be synthesised chemically by adding segments of natural genes to chemically synthesised DNA sequences. Alternatively, new primary sequences can be formed by using synthetic DNA sequences to enlarge natural genes or to replace segments of natural genes. The synthetic DNA or the hybrid DNA formed is subsequently inserted into a plasmid in order to synthesise the intended protein.

Molecular-biological modification: Thus, the primary sequence of calmodulin can be modified by methods known to the person skilled in the art in such a way that individual or several amino acids of the primary sequence are replaced by others. It is also possible to extend the primary sequence at the N- or C-terminal end by means of single amino acids, oligopeptides or whole proteins. It can be an advantage to produce only partial regions of the natural protein or the protein modified by molecular-biological methods, as is illustrated in Example 9. According to the invention, appropriate calmodulin that was genetically modified or a calmodulin fragment or a calmodulin derivative, which can be recognised by their activating effect on the PPIase activity, is especially preferred to activate the CaMAPs.

Post-translational modifications: Apart from the modification of the primary sequence described above, CaMAPs may be modified post-translationally. Thus, it can be for instance acetylated, methylated, ubiquitinised or phosphorylated. Numerous post-translational modification taking place in vivo can be accomplished by the person skilled in the art also in vitro. Thus, CaMAP can be phosphorylated in vitro as well as in vivo using different protein-serine/threonine and protein-tyrosine kinases and dephosphorylated using pleotropic protein phosphatases such as PP1gamma of PP2A (Eur. J. Biochem 269(2002)3619). Apart from the targeted in vitro phosphorylation of genetically produced calmodulin, comprising the defined phosphorylation of individual serines, threonines or tyrosines, (e.g. Zhang J G., Biochemical & Biophysical Research Communications. 222(2):439-444, 1996 or West et al., Protein Engineering. 2(4):307-11, 1988), calmodulin can be produced according to numerous instructions (e.g. Ho et al., Preparative Biochemistry. 16(4):297-308, 1987; or Caldwell and Haug in Analytical Biochemistry. 116(2):325-30, 1981) from various materials such as bovine brain, heart or testis. Depending on the source and the isolation method, the calmodulin obtained exhibits different post-translational modifications. Surveys of post-translational modifications of proteins are known to those skilled in the art and have been described in numerous publications, e.g.: Annals of the New York Academy of Sciences 663:48-62, 1992; Advances in Protein Chemistry. 37:247-334, 1985; Science. 198(4320):890-6, 1977; Current Opinion in Rheumatology. 14(3):244-9, 2002; Biomolecular Engineering. 18(5):213-20, 2001; International Journal of Biochemistry. 24(1):19-28, 1992). CaMAPs and CaMAP peptide fragments/derivatives that are post-translationally modified and, according to the invention, are particularly appropriate to activate the CaMAPs are recognisable by the activating effect of calmodulin on the PPIase activity of CaMAP.

Protein-chemical modifications: According to the invention, it can be an advantage to modify the CaMAP chemically. The methods and procedures necessary to this aim are known to those skilled in the art as protein-chemical methods. Besides the modification of lysines (Journal of Biological Chemistry. 273(43):28516-28523, 1998; Biochemica et Biophysica Acta. 844(2):265-9, 1985), the oxidation or carbethoxylation of calmodulin (Pharmacology. 26(5):249-57, 1983; Biochemistry. 17(19):3924-8, 1978), various chemical modifications of proteins are possible. Known techniques of chemical modification are described amongst others in: Current Opinion in Biotechnology. 10(4):324-30, 1999; Current Opinion in Chemical Biology. 5(6):696-704, 2001; Biochemistry-Russia. 63(3):334-44, 1998; Biotechnology & Applied Biochemistry. 26 (Pt 3):143-51, 1997; Biological Research. 29(1):127-40, 1996; Methods in Molecular Biology. 35:171-85, 1994; Methods in Molecular Biology. 32:311-20, 1994; Nature Cell Biology. 5(1):28-37, 2003). Chemically modified CaMAP or a CaMAP peptide fragment/derivative particularly suitable for the method according to the invention for the activation of CaMAPs is preferred and is recognisable by the activating effect on the PPIase activity of a CaMAP.

According to the invention, the term “appropriate CaMAP substrates” includes all substrates of the peptidyl-prolyl cis/trans isomerases. In particular, these are substances having a prolyl peptide bond. “Appropriate CaMAP substrates” in accordance with the invention are described in numerous articles of the technical literature known and available to the person skilled in the art, e.g.: Clinical Chemistry. 44(3):502-8, 1998; Analytical Biochemistry. 252(2):299-307, 1997; Biochemistry. 34(41):13594-602, 1995; Biochemistry. 30(25):6127-34, 1991; Biochemistry 30(25):6127-34, 1991; Journal of Molecular Biology. 271(5):827-37, 1997, or are cited in patent specifications such as CA2334812; EP0647713; WO0142245; EP0360029. Especially preferred CaMAP substrates, in accordance with the invention, are peptides showing a Xaa-Pro-Yaa-group, wherein Xaa designates preferably the amino acids Glu, Phe, or Leu, such as Suc-Ala-Phe-Pro-Phe-NHNp or Suc-Ala-Ala-Glu-Pro-Arg-NHNp.

“Appropriate amounts” of the components indicated in accordance with the method of the invention can be found amongst others in the Examples and, with regard to the CaMAP or CaMAP peptide fragment/derivative, calmodulin or calmodulin fragment/derivative as well as the CaMAP substrate, they are within the range from 0.01 μM to 10 mM in the reaction solution. A range of 0.1 μM to 1 mM is preferred. A value of 1 μM is particularly preferred.

The “appropriate” reaction solution of the invention is defined as buffer system that, besides water and buffer components, may also contain further components, such as those specified hereafter, and that ensures the identification of an effector of the clamodulin-dependent peptidyl-prolyl cis/trans isomerases.

The detection of the calmodulin-induced CaMAP activation is not obvious to the person skilled in the art (see Example 1). CaMAP FKBP38, for instance, tends to precipitate. This can falsify or prevent the determination of activity. Thus, the addition of calcium ions, which are usually used for calmodulin activation, causes, in concentrations higher than 5 mM, the proteins to precipitate in the measuring preparation. As shown in Examples 1, 2 and 3, it can be an advantage to use a protein without a hydrophobic binding anchor (as described in Example 9) in the activity assays. The detection of CaMAP activity via the complete protein proves to be more difficult due to precipitation.

The components contained in the reaction solution after mixing are preferably present in dissolved form. In order to prepare the reaction preparations in appropriate reaction vessels, the components have to be added while mixing. Mixing can already be achieved by appropriate pipetting techniques known to the person skilled in the art. In addition, the mixing effect can be enhanced mechanically, for example, by vortexing, swivelling or shaking.

Furthermore, it can be advantageous to incubate the reaction solution for a certain time, i.e. for 5 sec, 10 sec, 30 sec, 1 min, 2 min, 5 min, 10 min, or 20 min, after all necessary components have been mixed and before the reaction is started.

CaMAP effectors are easily identified in accordance with the invention. For this purpose, according to the invention, a constitutively active CaMAP can be used or the CaMAP can be activated with calmodulin, the corresponding fragments or derivatives. This activation modifies the catalytic center of the CaMAP in such a way that, under optimum catalysis conditions, the catalysis of typical CaMAP substrates is increased at least twofold. This at least 1.5-fold, preferably at least twofold increase of CaMAP activity is also characteristic for optimum catalysis conditions. To achieve optimum catalysis conditions, it is possible to use a wide range of buffers. In particular, a buffer with a pH value in the range of 5-10, more preferably in the range of 6-8 and particularly preferred in the range of 6.5-8.0 can be used. An appropriate buffer system is, for example, 20 mM HEPES buffer with pH 7.8. Further buffer solutions of the invention comprise, amongst others, TRIS/HCl, HEPES/NaOH or ammonium carbonate buffer in a final concentration of 5 mM to 200 mM.

The reaction temperature of the method of the invention can be chosen in the range from 0° C. to 30° C., preferably in the range from 5° C. to 15° C., particularly preferred, the temperature is at 8° C. Higher temperature can result in denaturation of the proteins, while a lower temperature will cause the reaction rate to decrease. In addition, the reaction mixture can contain protein stabilising substances, such as sucrose, sorbitol, or ethylene glycol, preferably in an amount of 200 to 500 mM and particularly preferred in an amount of 250 to 300 mM. Furthermore, it may be advantageous to add proteinase inhibitors, e.g. the “PI Complet” mixture obtainable from Roche®), in the amount range indicated by the manufacturer. As calmodulin in accordance with the invention can be obtained by adding bivalent ions such as Ca²⁺, chemicals added to the reaction preparation may mask the bivalent ion that is necessary for the activation of calmodulin only to the degree that the formation of an effective calmodulin is not inhibited. Example 11 illustrates how the activation of a CaMAP is achieved by adding a mixture of calcium ions and calmodulin to the assay preparation and how this activation can be inhibited by chelating the calcium ions. As shown in Example 5, effectors of activated CaMAPs may have a significant influence on the PPIase catalysis. The influence is considered significant if the activation of CaMAP is modified by at least 50% with the use of inhibitors possibly offsetting the calmodulin activation as illustrated in Example 5.

Affinity assays for the detection of CaMAP effectors: Effectors of CaMAPs can influence the conformation modification of CaMAP caused by calmodulin (Example 11) in such a way that the PPIase activity of the “activated CaMAP” is modified despite otherwise optimum reaction conditions. Thus, effectors can affect, for example, the binding site to the effective calmodulin or the calmodulin binding site to CaMAP. Inhibitors may also affect the binding sites for bivalent metal ions, which may be necessary for the activation of effective calmodulin, for example the binding sites for calcium ions. Inhibitors may, however, directly affect the PPIase catalytic center. Thus, the agent cyclosporin A can inhibit CaMAP Cyp40 (Swiss-Prot nomenclature: CYP4_HUMAN) significantly, as shown in Example 8. The agent FK506, however, can inhibit the CaMAP FKB38 (Swiss-Prot nomenclature: FKB8_HUMAN significantly, as shown in Example 5. Effectors of CaMAPs can be detected by using affinity assays known to those skilled in the art. Such affinity assays for the detection of a ligand binding may be designed differently. Examples can be found in patent specifications WO03018846; EP1202056; U.S. Pat. No. 6,281,006; WO0122084; CA2162568; U.S. Pat. No. 5,434,052; GB2300260; DE10030798A1; U.S. Pat. No. 5,773,225; NZ504112; HU0201142; HK1029376. In general, the detection methods can be classified under two types: (A) methods in which the effector affects the activation of CaMAP. The fact that the intensity of the interaction between effective calmodulin and CaMAP is affected, as shown in Examples 6 and 11, is characteristic of this method. And (B) methods in which the effector binds at or near the catalytic center. Binding assays where ligands of activated CaMAPs are detected due to the fact that they displace ligands already known from the catalytic center are characteristic of this method. Such an embodiment is illustrated in Example 7.

Activity assays for the detection of effectors of activated CaMAPs: Optimum methods for the detection of effectors of CaMAP activity can be determined by means of the effectors cyclosporin A and FK506 already known. To this end, CaMAP activity is activated by a sufficient amount of an effective calmodulin vis-à-vis an appropriate CaMAP substrate—using an appropriate PPIase activity test—in such a way that by adding a minimum amount of a known CaMAP inhibitor, a significant reduction of the CaMAP activity can be observed. In order to achieve an activation of CaMAPs using effective calmodulin, it can be advantageous to incubate CaMAP at 20° C. up to 20 minutes with the protein or protein fragment. It may, however, be advantageous to store a greater amount of CaMAP—that has already been activated by the addition of an appropriate amount of effective calmodulin—in aliquots after its preparation in such a way that the thus activated CaMAP is already available for activity measuring. To inhibit CaMAP activity via effectors, it may be advantageous to incubate CaMAP with an appropriate concentration of effective calmodulin in a first step, to add the effector after this first incubation and further to incubate the effector with the mixture. It may, however, be advantageous to change the order of the additions or to carry out a simultaneous incubation. When using constitutively active CaMAP, the addition of effective calmodulin can be omitted. The effector should be incubated with activated CaMAP for at least 1 second, preferably 300 seconds, but the incubation periods can be much longer to allow the observation of an effectuation. Appropriate CaMAP assays are all PPIase activity assays that allow a pre-incubation of the effector and activated CaMAP and enable the measuring of the effectuation by CaMAP effectors.

Effect of CaMAP inhibitors: The effects of effectors such as FK506 or CsA are not limited to the PPIase activity of activated CaMAPs. Thus, cyclosporin A inhibits PPIases belonging to the family of cyclophilins and FK506 inhibits PPIases belonging to the group of FKBPs (European Journal of Biochemistry. 216(3):689-707, 1993; Annual Review of Immunology. 10:519-60, 1992). Cyclosporin A as well as FK506 are currently used in human medicine as immunomedicaments to prevent the rejection of transplants. Both medicaments can cause significant side effects (e.g.: Seminars in Nephrology. 17(l):34-45, 1997). One possible side effect of these therapeuticals at cell level is the influence on the programmed cell death, designated as apoptosis by those skilled in the art. Apoptosis is necessary for normal embryogenesis and metamorphosis, tissue homeostasis as well as the functioning of the immune system in metazoes. At the microscopic level of the cell, apoptosis leads to the loss of cell connections and of microvilli, to chromatin condensation, DNA fragmentation, cytoplasmatic contraction and dense packing of mitochondries and ribosomes. Membrane vesicles are formed in which the endoplasmatic reticulum fuses with the cytoplasmatic membrane and subdivides the cell into several membrane-bound vesicles, named apoptotic bodies. In general, the latter are taken up and degraded by adjacent cells. Up to date, a number of apoptosis activating factors have been identified. Apoptosis can be induced by cytotoxic agents. It seems, however, that the natural and genetically programmed apoptosis depends on the initiation of a signal pathway through ligand binding to specific receptors on the cell surface. [R. E. Ellis et al., Annu. Rev. Cell Biol. 7 (1991) 663-698; S. Cory, Nature 367 (1994) 317-318; S. J. Martin and D. R. Green, Cell 82 (1995) 349-352; M. Tewari et al., J. Biol. Chem. 270 (1995) 18.738-18.741; C. D. Gregory (ed.) Apoptosis and the Immune Response Wiley-Liss., New York, 1995]. There are known examples in the literature where the inhibitors of CaMAP activity already known induce apoptosis (Cyclosporin A: International Journal of Molecular Medicine. 7(4): 431-437, 2001; Anticancer Research. 20(5B):3363-3373, 2001; Scandinavian Journal of Immunology. 56(4):353-360, 2002; FK506: Life Sciences. 66(23):2255-2260, 2000; Clinical & Experimental Immunology. 125(1):19-24, 2001) but also inhibit apoptosis (Scandinavian Journal of Immunology. 56(4):353-360, 2002; American Journal of Respiratory & Critical Care Medicine. 165(4):449-455, 2002; Carcinogenesis. 21(11):2027-2033, 2000; FEBS Letters. 447(2-3):274-276, 1999; FK506 Brain Research. 826(2):210-219, 1999; British Journal of Pharmacology. 126(5):1139-1146, 1999; NeuroReport. 9(9):2077-2080, 1998). One cause of the contradictory effect of the two inhibitors at cell level can be seen in the influence of further PPIases that cannot be activated with effective calmodulin. In human cells, more than 5 different PPIases are cyclosporine A-sensitive, and more than 5 PPIases are susceptible to FK506. That specific CaMAP inhibitors should cause apoptosis in human cells, is shown by a published experiment related to the depletion of CaMAP FKBP38 by means of siRNA (Nature Cell Biology 5(2003)1). That is why the method of the invention further provides a new method of identifying and producing effectors for the development of therapies aiming at purposefully inducing apoptosis in which CaMAPs are involved. Such means and therapies are relevant with regard to all diseases that can be treated by selective destruction of cells. This applies, in particular, to tumour diseases. The selective therapeutic induction of apoptosis may also be advantageous to influence immunogenicity of cells as is described, e.g. in U.S. Pat. No. 5,922,589.

The embodiment of the invention also comprises a method for screening and/or producing an effector of a CaMAP consisting of the steps of
(a) mixing appropriate amounts of a CaMAP or a CaMAP peptide fragment/derivatives with an appropriate amount of calmodulin or a calmodulin fragment/derivative in an appropriate reaction solution with and without a sample containing a single or a multitude of compounds which are candidates for an inhibitor or an activator;
(b) adding an appropriate amount of an appropriate CaMAP substrate;
(c) measuring CaMAP activity; and
(d) detecting that the sample
   (i) exhibits inhibitory activity if the CaMAP activity in the reaction solution with the sample is lower than in the reaction solution without the sample; or
   (ii) exhibits activating activity if the CaMAP activity in the reaction solution with the sample is higher than in the reaction solution without the sample.

The method in accordance with the invention includes the embodiments of steps (a) to (d) of the method for the determination if an effector is an inhibitor or an activator, wherein a sample is examined instead of an effector. “Sample” denotes all natural or artificial samples that contain candidates for inhibitors or activators of the given enzyme and which can be tested in the methods of the invention. These include homogenous solutions of a molecule as well as mixtures of several molecules. The molecules that can be screened in the process comprise the molecules of the embodiment described above which are characterised as effectors. The samples can be obtained from natural sources or can be produced synthetically. Samples can be obtained, for example, from molecule libraries such as existing libraries for oligopeptides or natural compounds. Furthermore, samples can be obtained by decomposition of biological material, e.g. live material or formerly live material or can originate from supernatants of microorganism cultures. The samples can be in form of a crude extract or crude supernatant or in a purified form that can be chosen ad lib. The extracts or supernatants can be fractionated for purification. Numerous techniques answering that purpose are available to those skilled in the art, such as differential precipitation, gradient centrifugation, chromatographical techniques, etc. The biological material comprises all organisms with the material either cultivated or obtained from nature.

In the context of the present invention, “screening” denotes the assay of a multitude of samples containing a single or a multitude of compounds which are candidates for inhibitors or activators of the given enzyme with the aim to identify inhibitors and activators of CaMAP. Generally, “screening” denotes a method for examining a multitude of samples for a particular characteristic wherein it is usually not known beforehand how the samples will react with regard to the characteristic that is to be assayed.

Another aspect of the present invention allows the quantification of effectors in biological materials or samples. CaMAP effectors can be found in biological materials as a result of various causes. Besides gene-encoded effectors that are intrinsically present in cells, effectors can also be introduced into biological materials—where they can be detected—by contamination with other biological materials, for example by an infection with bacteria or via the uptake of food—in particular when the intestinal absorption is defective. Effectors can, however, also be intentionally introduced into biological is materials as medicaments—either directly as agent or indirectly as precursor substance. The quantification of these CaMAP effectors can be useful for achieving an optimum therapy regime via the established bioavailability if the effectors are administered therapeutically. If specific concentrations of CaMAP effectors intrinsically present in cells are characteristic of specific states of these biological objects and it is possible to gather useful information from these particular states, it is advantageous to quantify their concentration. When quantifying CaMAP effectors, it may be an advantage to determine their concentration in form of a threshold assay. After defining a normal range describing the concentration of the CaMAP effectors in the biological sample of state (A), it is possible to infer from a significant deviation of the concentration of the CaMAP effector from this normal value a change from state (A) to state (B) of the biological sample. Examples 6 and 7 illustrate characteristic applications in accordance with the invention.

Moreover, the invention relates as a preferred embodiment to a method that comprises the above-identified steps (a) to (d) and, in addition, the following step:
e) fractioning of the sample for which inhibitory or activating activity was detected in step (d) and repeating of steps (a) to (d) until the inhibitor or activator contained in the sample is present in purified form.

In a further preferred embodiment, the CaMAPs of the method according to the invention are selected from the group consisting of human CaMAPs such as FKBP36, FKBP37.7, FKBP44, FKBP51, FKBP52 and Cyp40 and enzymes that are listed in the “Swiss-Prot” database,—which is accessible via the following URL: http://us.expasy.org/sprot/—, corresponding to the denotation used in this database under FKBP66, FKBP42, AIP_HUMAN, AIP_CERAE, AIP_MOUSE, AIPL1_HUMAN, AILP1_RAT, AILP1_MOUSE, AILP1_RABIT, FKB8_HUMAN, FKB8_MOUSE, FKB5_HUMAN, FKB5_MOUSE, FKB4_HUMAN, FKB4_MOUSE, FKB4_RABIT, FKB7_WHEAT, CYP4_BOVIN and CYP4_HUMAN.

In a further preferred embodiment, the calmodulin or the calmodulin fragment/derivative 10 of the invention and accessible in the “Swiss-Prot” database under the denotation of this database, which is given below, is selected from the group consisting of:
CALM_ACHKL (P15094), CALM_BLAEM (Q9HFY6), CALM_CANAL (P23286), CALM_CAPAN (P93087), CALM_CHLRE (P04352), CALM_DICDI (P02599), CALM_DROME (P07181), CALM_ELEEL (P02594), CALM_EMENI (P19533), CALM_EUGGR (P11118), CALM_FAGSY (Q39752), CALM_HELAN (P93171), CALM_HORVU (P13565), CALM_HUMAN (P02593), CALM_KLULA (060041), CALM_LYCES (P27161), CALM_LYTPI (P05935), CALM_MAGGR (Q9UWF0), CALM_MAIZE (P41040), CALM_MALDO (P48976), CALM_MEDSA (P17928), CALM_METSE (P02596), CALM_NEUCR (Q02052), CALM_ORYSA (P29612), CALM_PARTE (P07463), CALM_PATSP (P02595), CALM_PHYIN (P27165), CALM_PLAFA (P24044), CALM_PLECO (P11120), CALM_PNECA (P41041), CALM_PYUSP (P11121), CALM_SCHPO (P05933), CALM_SOLTU (P13868), CALM_SPIOL (P04353), CALM_STIJA (P21251), CALM_STRPU (P05934), CALM_STYLE (P27166), CALM_TETPY (P02598), CALM_TETTH (Q05055), CALM_TRYBB (P04465), CALM_TRYCR (P18061), CALM_WHEAT (P04464), CALM_YEAST (P06787), Q9UWF0, Q02052, P19533, AAL89686, Q7M510, Q96TN0, P27165, AAG01043, P02593, Q7T3T2, Q40302, O02367, Q95NR9, Q9UB37, AAH54805 AAH54973, AAL02363, AAH59427, AAH59500, AAH54600, AAH53150, AAH50926, AAH45298, AAH44434, AAP88918, AAP35501, AAP35464, BAC56543, AAC83174, AAD55398, AAC63306, AAD45181, AAH21347, BAC40168, BAB28631, BAB28319, BAB28116, BAB23462, AAH58485, AAH51444, AAH47523, P07181, Q7QGY7, Q8STF0, AAO25039, AAM50750, AAK61380, BAB89360, O94739, P02594, Q9D6G4, O16305, Q96HK3, P11120, O96102, P21251, Q9U6D3, Q8X187, O93410, AAR10240, P11121, Q9XZP2, Q42478, AAQ01510, P17928, P93171, O97341, O96081, AAD10244, AAM81203, AAA34238, AAA34014, AAA34013, P02596, P93087, Q43699, CAD20351, BAB61916, BAB61915, AAF65511, P02595, P59220, P27162, Q93V8, Q39447, Q94801, AAQ63462, AAQ63461, AAM81202, BAB61918, BAB61917, BAB61914, BAB61913, BAB61912, BAB61911, BAB61910, BAB61909, AAG27432, AAG11418,
   wherein these sequences or sequences that are similarly appropriate can easily be found in biochemical databases—which are continuously updated and expanded with new entries—using sequence analysis programs, such as BLAST.

In a further preferred embodiment, the appropriate reaction solution of the method in accordance with the invention contains bivalent ions selected from the group consisting of Zn²⁺, Cu²⁺, Co²⁺, Ni²⁺, Mn²⁺, Ca²⁺ and/or Mg²⁺. In a preferred embodiment, the appropriate reaction solution contains the bivalent ions of the invention in a concentration of 0.1 to 20 mM. Particularly preferred is a concentration of the bivalent ions at a concentration of 2.5, 3, 3.5, 4, 4.5, 5, 5.5, 6 or 6.5 mM. According to the invention, the bivalent ions can be present individually or in any combination in the reaction solution. Optionally, further ions, such as Na⁺, K⁺, Li⁺, can be added in a concentration of 0.5 to 100 mM of the reaction solution.

In a further preferred embodiment, the appropriate reaction solution of the method in accordance with the invention has a pH value of between pH 5 and pH 10. A pH value of 6, 6.25, 6.5, 6.75, 7, 7.25, 7.5, 7.75, 8 or 8.25 is especially preferred.

Additionally, a further embodiment of the invention relates to a method for the identification and/or the production of an effector of a CaMAP consisting of the steps
(a) mixing appropriate amounts of a constitutively active CaMAP in an appropriate reaction solution with and without effector;
(b) adding an appropriate amount of an appropriate CaMAP substrate;
(c) measuring CaMAP activity; and
(d) detecting that the effector is
   (i) an inhibitor if the CaMAP activity in the reaction solution with the effector is lower than in the reaction solution without the effector; or
   (ii) an activator if the CaMAP activity in the reaction solution with the effector is higher than in the reaction solution without the effector.

The expression “constitutively active” refers to the modification of a CaMAP via the application of either one or more of the methods described above in such a way that it achieves the enzymatic activity of a CaMAP without the presence of calmodulin. These methods according to the invention allow, under optimum conditions, an at least 1.5-fold increase of the activity of a CaMAP, and, in accordance with the invention, particularly preferred an at least 2-fold increase of said activity. Strategies to obtain a constitutively active enzyme are known to the person skilled in the art and have been described repeatedly, cf.: Journal of Biological Chemistry. 272(6):3223-3230, 1997; Journal of Neurobiology. 52(1):24-42, 2002 and FEBS Letters. 503(2-3):185-188, 2001. The use of a constitutively activated CaMAP is particularly preferred for the method according to the invention.

Furthermore, a preferred embodiment of the invention is a method where steps (a) and (b) are interchanged.

The method of the invention further allows the detection of the effector by spectroscopic or radioactive methods. Spectroscopic methods within the meaning of the invention, are known to those skilled in the art and comprise amongst others CD spectroscopy, fluorescence spectroscopy, absorption spectroscopy. Further, it is possible to detect effectors in accordance with the invention by using mass spectrometric methods, such as MS-MALDI, ligand binding methods, such as Biacore, or structural methods, such as NMR techniques. Radioactive methods are known to the person skilled in the art and are mentioned in Examples 6, 7 and 10.

The method of the invention is preferably a high-throughput method.

Effectors of the invention that can be identified by means of the method of the invention are, for example, cycloheximide derivatives having the general formula 1: 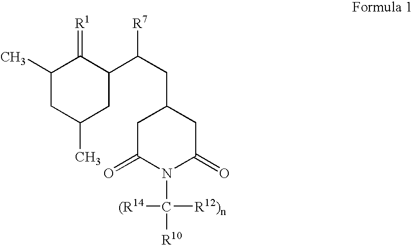
in which n is an integer from 1 to 20; R¹² independently is a hydrogen atom, an alkyl residue or an aryl residue,
R¹ is selected from an oxygen atom, a sulfur atom, or the groups NR², NOR² and N—NR²R³, wherein
   a) R² and R³, independently from each other, are a hydrogen atom, aryl or alkyl, respectively, which can optionally be interrupted by O, S, NH, NR⁵, aryl, heteroaryl, cycloalkyl, heterocycloalkyl or can optionally be substituted by R⁶, or
   b) R² and R³ together are C₁-C₆-alkylene, which can optionally be interrupted by O, S, NH, NR⁵, aryl, heteroaryl, cycloalkyl or heterocycloalkyl or can optionally be substituted by R⁶, wherein
      R⁵ is an alkyl residue or an aryl residue,
      R⁶ stands for a hydrogen atom, alkyl, aryl, OR⁵, C(O)OR⁵, CN, F or Cl, wherein R⁵ is defined as above,
R⁷ is a —OH, —OR⁹, —OC(O)R⁹, —OC(S)R⁹, —OC(O)NHR⁹ or —OC(S)NHR⁹ residue, wherein R⁹ is an aryl residue which can optionally be interrupted by O, S, NH, NR⁵, aryl, heteroaryl, cycloalkyl or heterocycloalkyl or can optionally be substituted by R⁶ as defined above, or alternatively
R⁹ is an aryl residue which can optionally be interrupted by O, S, NH, NR⁵ or can optionally be substituted by R⁶ as defined above
R¹⁰ is a —NHR², —NR²R³, —C(O)OR², —C(S)OR², —C(O)NR²R³, —CN, —NR²C(O)NR²R³, —OC(O)NR²R³, —NR²C(S)NR²R³, —OC(S)NR²R³, or OR², C(O)NHR¹¹ residue, wherein R² and R³ are defined as above,
R¹¹ stands for an amino acid residue or an oligopeptide residue and
R¹⁴ is an alkyl residue or an aryl residue.

Alternative compounds in accordance with the invention having the general formula 1 differ from the above cited compounds in that they have a hydrogen atom at position R¹⁴ and, between the adjacent carbon atom and R¹⁵ form an ether in the following manner (Formula 2), wherein R¹⁵ is selected from alkyl residue, aryl residue or hydrogen atom:

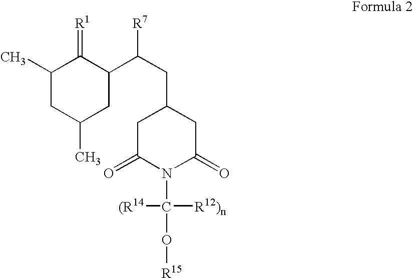

In comparison to the substances that are described, for example, in WO 00/26188, the compounds of the invention in which R¹⁴ is an alkyl residue have the advantage of enhanced lipophilicity and, in the case of ether according to formula 2, the advantage of enhanced stability.

Preferred embodiments of the compound according to the invention having the above-identified formula 1 are compounds for which the following applies:
(a) n=1, 2, 3; R¹=O; R⁷=OH, O(CHR¹²)ₙR¹⁰, OC(O)CH₃; R¹⁰=C(O)OCH₃, C(O)OC₂H₅, CN, C(O)NH₂,
(b) n=3-10; R¹=O; R⁷=OH; R¹⁰=C(O)NHR¹¹, R¹¹=amino acid residue, oligopeptide residue,
(c) n=1, 2, 3; R¹=O; R⁷=OH, O(CHR¹²)ₙR¹⁰; R¹⁰=C(O)OCH₃, C(O)OC₂H₅, CN, C(O)NH₂,
(d) n=1, 2, 3; R¹=NOH, N—NHPh, N—NHCH₃, N-alkyl, N-benzyl; R⁷=OH, O(CHR¹²)ₙR¹⁰; R¹⁰=C(O)OCH₃, C(O)OC₂H₅, CN, C(O)NH₂,
(e) n=1, 2, 3; R¹=O; R⁷=OH, O(CHR¹²)ₙR¹⁰, OC(O)NH-alkyl, OC(O)NH-cycloalkyl, OC(O)NH-aryl; R¹⁰=C(O)OCH₃, C(O)OC₂H₅, CN, C(O)NH₂.

Preferred compounds of the present invention are the following compounds 1 to 39 according to formula 1:

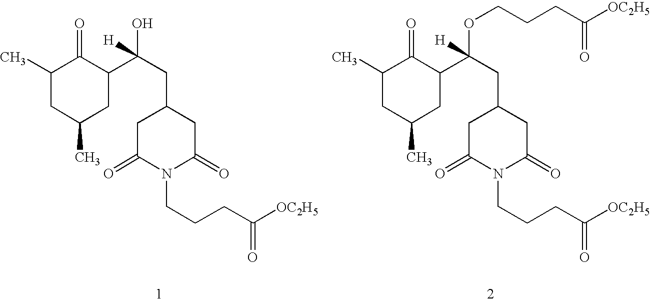

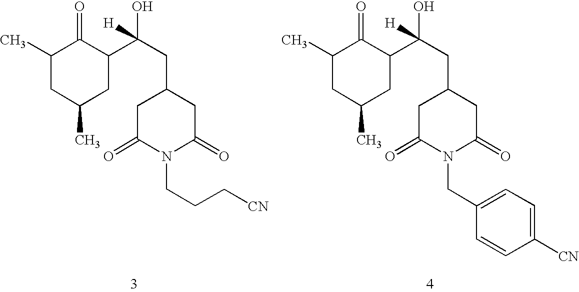

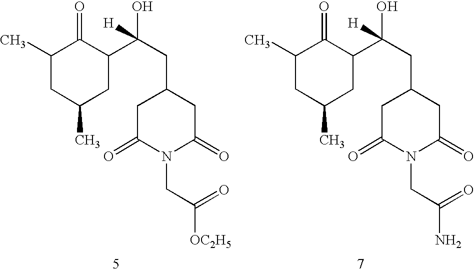

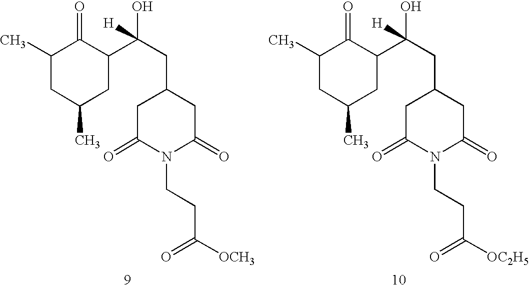

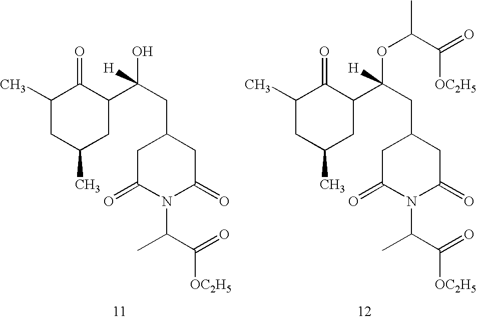

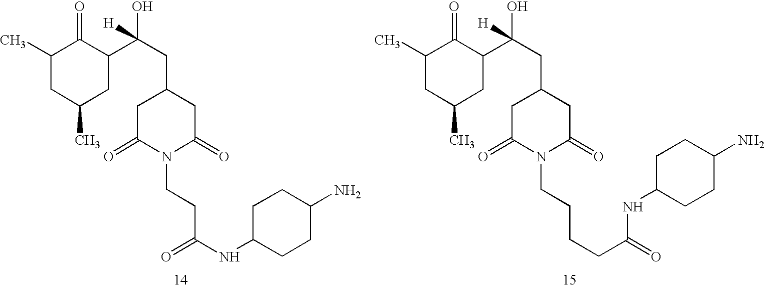

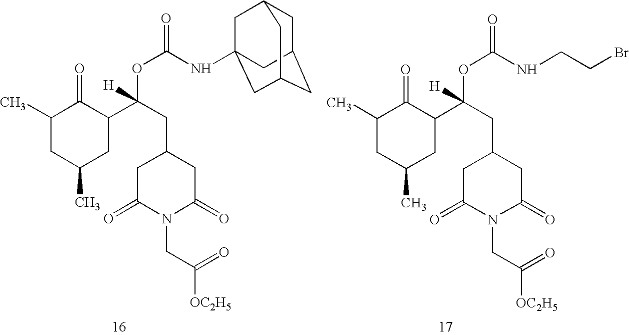

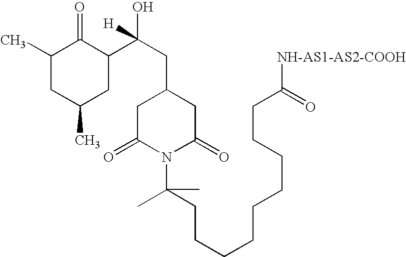

| compound | amino acid residue AS1 | amino acid residue AS2 |
|---|---|---|
| 18 | alanine | alanine |
| 19 | valine | alanine |
| 20 | tryptophan | alanine |
| 21 | isoleucine | alanine |
| 22 | methionine | alanine |
| 23 | glycine | alanine |
| 24 | alanine | valine |
| 25 | valine | valine |
| 26 | tryptophan | valine |
| 27 | isoleucine | valine |
| 28 | methionine | valine |
| 29 | glycine | valine |

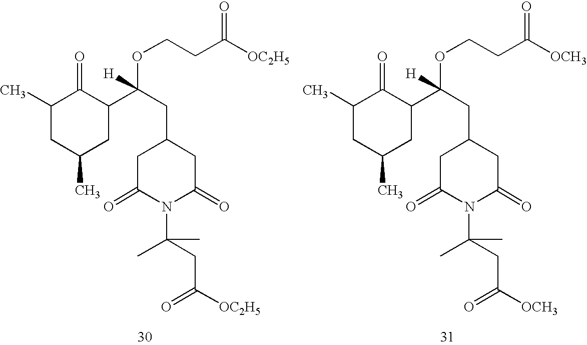

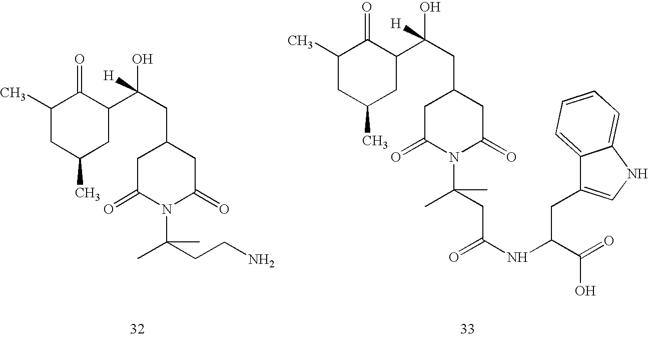

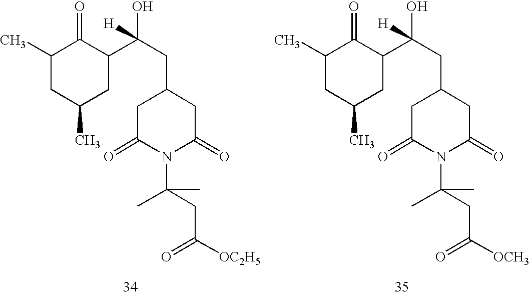

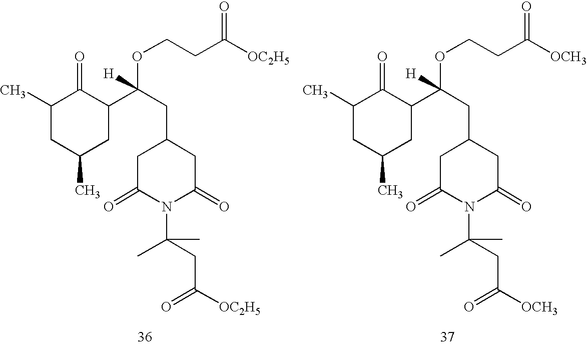

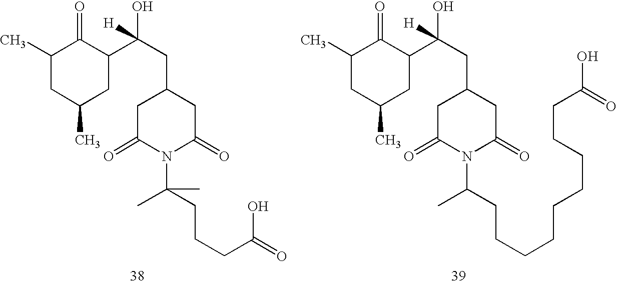

In the above formulas of the compounds in accordance with the invention as well as in the following, alkyl refers to a linear or branched alkyl residue, in particular C₁-C₈-alkyl, or alkyl substituted by alkyl, aryl, heteroaryl, halogen, and particularly preferred F, CN, NO₂, S, O, C(O). In the above formulas of the compounds of the invention and in the following, cycloalkyl refers, in particular, to C₄-C₇-cycloalkyl, or bi- and tri-cyclical systems, which can be substituted by alkyl, aryl, heteroaryl, halogen, and particularly preferred by F, CN, NO₂, S, O, C(O). Aryl refers, in particular, to phenyl or aryl substituted by alkyl, aryl, heteroaryl, halogen, and in particular F, CN, NO₂, C(O) and heteroaryl, in particular six-membered aromatic hydrocarbons containing nitrogen or five-membered aromatic hydrocarbons containing nitrogen, oxygen or sulfur. Oligopeptide residues, in particular, refer to residues from 2-5 condensed amino acids. Halogen refers to F, Cl, Br and/or I.

The compounds in accordance with the invention are, for example, produced in the following manner known to the person skilled in the art by
a) reacting cycloheximide and appropriate derivatives in an appropriate solvent, such as anhydrous dimethylformamide, at room temperature with halogen alkyl compounds and an appropriate base, such as K₂CO₃, to the corresponding N-alkyl or N,O-bisalkyl compounds;
b) reacting cycloheximide and appropriate derivatives in an appropriate solvent, such as anhydrous acetone, at room temperature with halogen alkyl compounds and an appropriate base, such as K₂CO₃, and while adding catalytic amounts of crown ethers, such as 18-crown-6-ether, to the corresponding N-alkyl or N,O-bisalkyl compounds;
c) reacting cycloheximide and appropriate derivatives in an appropriate solvent, such as anhydrous acetone, at boiling heat with halogen alkyl compounds and an appropriate base, such as K₂CO₃, and while adding catalytic amounts of crown ethers, such as 18-crown-6-ether, to the corresponding N-alkyl or N,O-bisalkyl compounds;
d) reacting cycloheximide and appropriate derivatives in an appropriate solvent, such as anhydrous dimethylformamide, at room temperature with halogen alkyl compounds bound to carrier materials (standard resins for solid-phase reactions) and an appropriate base, such as K₂CO₃, and while adding catalytic amounts of crown ethers, such as 18-crown-6-ether, to the corresponding N-alkyl compounds and cleaving off the carrier by conventional methods;
e) reacting cycloheximide in an appropriate solvent, such as pyridine, at room temperature with carboxylic acid anhydrides to the corresponding esters;
f) reacting cycloheximide derivatives in an appropriate solvent, such as a mixture of pyridine/H₂O (2:1), at room temperature with hydroxylamine hydrochlorides to the corresponding oximes;
g) reacting cycloheximide derivatives in an appropriate solvent, such as anhydrous methanol, at room temperature with hydrazines to the corresponding hydrazones;
h) reacting cycloheximide derivatives in an appropriate solvent, such as anhydrous methanol, at room temperature with primary amines to the corresponding azomethines;
i) reacting cycloheximide derivatives in an appropriate solvent, such as methylene chloride, at room temperature with isocyanates and an appropriate catalyst, such as HCl, to the corresponding urethanes.

The invention further relates to a method comprising the above-identified steps (a) to (e) and, in addition, the following step:
(f) formulating the effector identified and/or produced with a pharmaceutically acceptable carrier or solvent.

In a further preferred embodiment, the effector identified and/or produced in accordance with the invention, is formulated, optionally, in combination with a “pharmacologically acceptable” carrier and/or solvent. Examples of particularly appropriate carriers that are pharmacologically acceptable are known to the person skilled in the art and comprise buffered saline solutions, water, emulsions, such as oil/water emulsions, different kinds of detergents, sterile solutions, etc.

The present invention also comprises the use of the effector identified and/or produced in accordance with the invention for the production of a medicament for the treatment of tumour diseases. Tumour diseases treated with the medicament in compliance with the invention comprise breast cancer, ovarian cancer, bronchial carcinoma, colon carcinoma, melanoma, bladder carcinoma, gastric carcinoma, head and neck tumours, brain tumours, cervical carcinoma, prostatic carcinoma, testicular carcinoma, bone tumours, carcinoma of kidney, pancreatic carcinoma, esophageal carcinoma, malignant lymphoma, Non-Hodgkin lymphoma, Hodgkin lymphoma and thyroid lymphoma.

Medicaments according to the invention comprising the pharmacologically acceptable carriers mentioned above can be formulated by using known conventional methods. These medicaments can be administered to a subject in an appropriate dose. The administration can be oral or parenteral, for example intravenous, intraperitoneal, subcutaneous, intramuscular, local, intranasal, intrabronchial or intradermal, or via an arterial catheter. The kind of dosage will be determined by the attending physician in accordance with the clinical factors. The person skilled in the art knows that the kind of dosage depends on various factors, such as body height and weight, body surface, age, gender or general health of the patient, but also on the particular medicament that is to be administered, the duration and type of administration as well as on other medicaments that are possibly administered simultaneously. A typical dose can be in the range of between 0.01 and 10,000 μg; doses below and above this exemplary range are possible, especially when taking into account the above-identified factors. Given regular administration of the pharmaceutical formulation of the invention, the dose should, in general, be within a range of between 10 ng and 10 mg units per day or application interval. If the composition is administered intravenously, the dose should be within a range of between 1 ng and 0.1 mg units per kilogram body weight and minute.

The composition of the invention can be administered locally or systemically. Preparations for a parenteral administration comprise sterile aqueous or non-aqueous solutions, suspensions and emulsions. Examples of non-aqueous solvents are propylene glycol, polyethylene glycol, vegetable oils, such as olive oil, and organic ester compounds, such as ethyloleate, that are appropriate for injection. Aqueous carriers comprise water, aqueous alcoholic solutions, emulsions, suspensions, saline and buffered media. Parenteral carriers comprise sodium chloride solutions, Ringer's dextrose, dextrose and sodium chloride, Ringer's lactate and bound oils. Intraveneous carriers comprise amongst others liquid additives, nutrient additives and electrolyte additives (e.g. additives based on Ringer's dextrose). The medicament of the invention may further comprise preservative agents and other additives, such as antimicrobial compounds, antioxidants, complexing agents and inert gases. Furthermore, depending on the intended use, compounds, such as interleukines, growth factors, differentiation factors, interferones, chemotactic proteins or an unspecific immunomodulating agent may be contained.

CaMAP effectors identified according to the invention, i.e. substances that are suitable for activating, inhibiting or stabilising CaMAPs and the specific effect of which on the CaMAP activity in biological objects results in a predominantly therapeutical influence on the pathobiochemical processes in these biological objects, are appropriate therapeuticals for such processes. If therapeutically usable CaMAP effectors are gene-encoded, it may be an advantage to introduce the sequence information that is necessary for synthesis in the organism to be treated therapeutically into this organism by using gene therapeutic methods known to those skilled in the art. It can, however, also be helpful to produce the agent (CaMAP effector) useful for the therapy as precursor on the basis of which the active substance (agent) will form either at the actual site of effect or on the way to the site of effect. The reasons in favour of this method can be manifold. Thus, with instable agents, the administration of the agent in form of a precursor allows to increase the stability and, thus, to render the agent bioavailable. Specific modifications of the substance can, however, also be suitable for modifying solubility characteristics in a desired way or allow that biological barriers, which can inhibit the penetration of an agent to the site of effect, are rendered directionally penetrable for the modified substance or its precursor. Furthermore, it is comprised that to enhance the pharmacological characteristics of the effector identified in accordance with the invention, the effector is further modified in order to exhibit modified organ specificity, enhanced activity, increased toxicity for tumour cells (an enhanced therapeutic index), decreased side-effects, delayed start of the therapeutic effect or the duration of the therapeutic effect, modified pharmacokinetic parameters (resorption, distribution, metabolism or excretion), modified physicochemical parameters (solubility, hygroscopic properties, colour, taste, odour, stability, configuration), enhanced general specificity, organ/tissue specificity, and/or an optimized administration form and route, which can be achieved by esterifying carboxyl groups, hydroxyl groups with carboxylic acids, hydroxyl groups, for example, to phosphates, pyrophosphates, sulfates, “hemisuccinates” or by formation of pharmaceutically acceptable salts, pharmaceutically acceptable complexes, or the synthesis of pharmacologically active polymers, or the introduction of hydrophilic groups, the introduction or the exchange of substituents in aromatics or side chains, the modification of the substituent pattern or by modification through introduction of isosteric or bioisosteric groups or by synthesis of homologous compounds, or introduction of branched side chains, by conversion of alkyl substituents into cyclic analogues, by derivatization of hydroxyl groups into ketals or acetals, by N-acetylation into amides, phenylcarbamates, by the synthesis of Mannich bases or imines, or by transformation of ketones, aldehydes into Schiff bases, oximes, acetals, ketals, enolester, oxazolidines, thiazolidines or combinations thereof.

The invention comprises the use of the effector identified and/or produced in accordance with the invention for the production of a medicament for the inhibition or attenuation of transplant rejection. Further, the use of the effector identified and/or produced in accordance with the invention comprises the production of a medicament for influencing neurodegenerative diseases/disorders such as: Alper's disease, Alzheimer's disease, Batten disease, Cockayne syndrome, corticobasal ganglionic degeneration, Huntington's disease, idiopathic Parkinson's disease, Lewy-Body disease, motor neurone disease, multiple systemic atrophy, multiple sclerosis, olivopontocerebellar atrophy, Parkinson's disease, postpoliomyelitis syndrome, prion disease, progressive supranuclear paralysis, Rett syndrome, Shy-Drager syndrome, and tuberous sclerosis.

In a further preferred embodiment, the present invention relates to a kit comprising the CaMAP according to the invention or a CaMAP peptide fragment/derivative, calmodulin according to the invention or a calmodulin fragment/derivative, one or more buffer solutions, and one or more substrates. Optionally, further, an instruction for carrying out one or more of the methods described above.

Such instruction contains the descriptions included in the present description of the invention, which enable the user to apply the method(s) of the invention. Additionally, the instruction can include indications pertaining to the state of the art and facilitating the carrying out of particular techniques.

The present application document cites a number of documents. Herewith, by reference all of the documents cited herein (including any kind of description of manufacturers, instructions etc.) are made subject matter of the present application; it is, however, not conceded that any of the cited documents is actually pertaining to the state of the art of the present invention.

The Figures show:

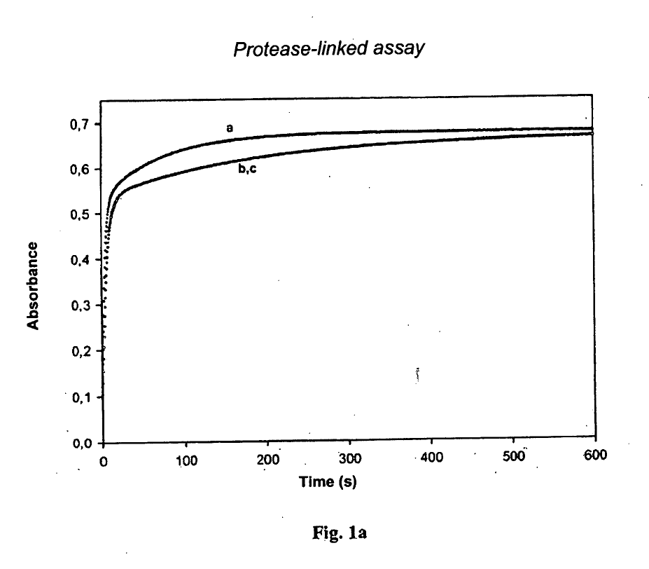

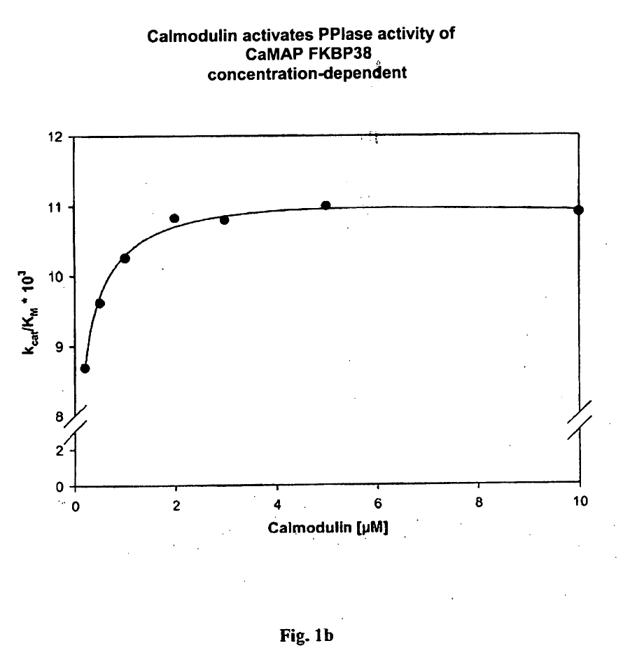

FIG. 1 FIG. 1a shows the isomer-specific p-nitroanilide release at 390 nm in presence (a) and absence (b) of 2 μM calmodulin as well as without FKBP38 (c). Reaction curves b and c are nearly identical and correspond to the uncatalysed spontaneous cis/trans isomerization. FIG. 1b shows the dependency of CaMAP activity on CA²⁺/calmodulin concentration. The carrying out of the experiment is described in Example 1. With concentrations>5 mM of calcium chloride, precipitation of the sample was observed.

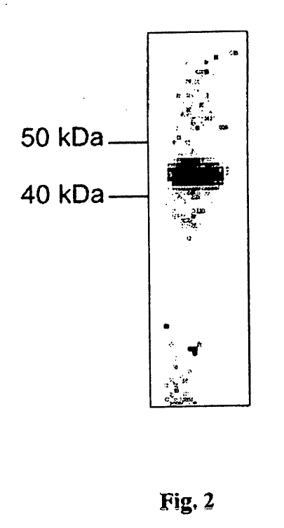

FIG. 2 FIG. 2 shows a comprehensive view, obtained using SDS-PAGE and phosphoimager, of chromatographically purified CaMAP FKBP38 that was produced according to Example 10.

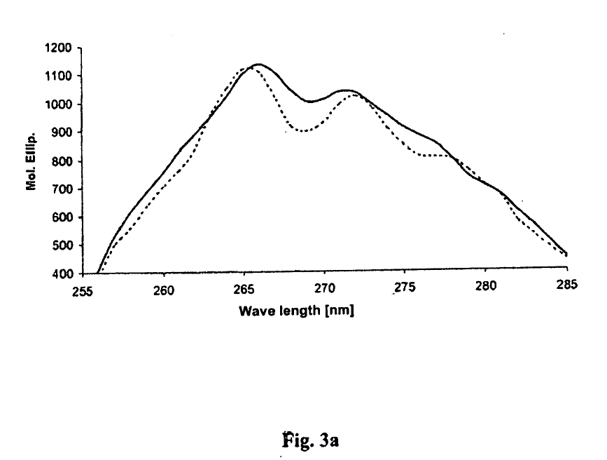

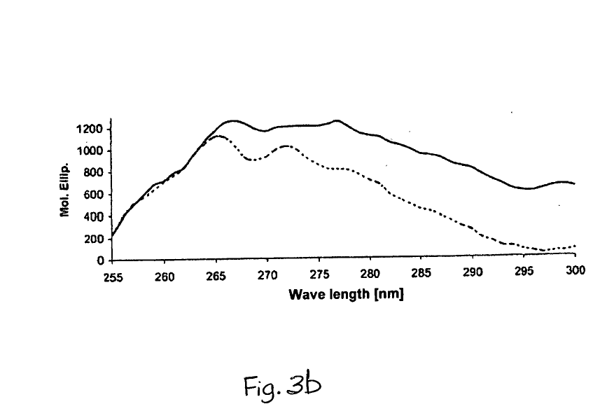

FIG. 3 FIG. 3 shows the CD spectrographically detected structure modification (CD spectra) according to Example 11. The continous line in FIG. 3a corresponds to the overall spectrum modification of solution A (5 mM CaCl₂, 10 μM FKBP38 in CD buffer) and B (20 μM calmodulin (bovine brain, Sigma) in CD buffer) in the different chamber sections. A solution of 10 mM HEPES buffer and 5 mM calcium chloride with a pH value of 7.5 was used as CD buffer. The dotted line in FIG. 3a shows a characteristic spectrum occurring after mixing the two solutions. After adding 1 M EGTA to the mixture, the spectrum characteristic of the non-interacting proteins calmodulin and FKBP38 is obtained (continuous line).

The dotted line in FIG. 3b shows the characteristic spectrum after mixing the two solutions. The spectrum of the continuous line was obtained after increasing the calcium chloride concentration to 10 mM.

The following Examples illustrate the present invention without limiting its scope to these Examples:

### EXAMPLE 1

### CaMAP Activation with Calmodulin

The following Example shows the characteristic dose-depending activation of PPIase activity of a CaMAP with calmodulin.

The human CaMAP FKBP38 (synonyms: FKBP8_HUMAN; Swiss-Prot-No.: Q14318) was produced molecular-biologically, as described in Example 9, and stored in aliquots of 100 μl with a protein concentration of 0.83 mg/ml at −80° C. Just before measuring the activity, one of these aliquots was thawed and subsequently stored at 4° C. Furthermore, commercially available calmodulin isolated from bovine brain (Sigma; purchase order no.: P2277), which had been stored at −80° C., was thawed just before measuring the activity and stored at 4° C. Suc-Ala-Phe-Pro-Phe-pNA (Bachem; purchase order no.: L-1400) was used as CaMAP substrate. Alpha-chymotrypsin isolated from bovine pancreas (Merck KG; purchase order no.: 102307) was used as isomer specific auxiliary enzyme. The following working solutions were prepared just before measuring and stored at 4° C.:
solution A: 55 mM HEPES buffer, pH 7.8; 1 mM DTT, 0.5% glycerol
solution B: 20 mg of the peptide substrate dissolved in 1 ml DMSO
solution C: 20 mg chymotrypsin dissolved in 200 μl of solution A
solution D: FKBP38 (100 μM, diluted in solution A)
solution E: calmodulin (300 μM, diluted in solution A)
solution F: calcium chloride (1 M in solution A)

For the kinetic determination of CaMAP activity, a computer assisted diode-array spectrometer (Hewlett Packard) with a cuvette temperature of 4° C. is used.

A typical measuring preparation contained 1 μl of solution B; 1 μM FKBP38, 2 μM calmodulin and 0 to 10 mM calcium chloride. The total volume was adjusted to 1200 μl with solution A. After preincubating for 5 minutes for the formation of the activated CaMAP, activity measuring was started by adding of 3 μl of solution C. FIG. 1a shows the isomer-specific p-nitroanilide release at 390 nm in presence (a) and absence (b) of 2 μM calmodulin as well as without FKBP38 (c). Reaction curves b and c are nearly identical and correspond to the uncatalysed spontaneous cis/trans isomerization. FIG. 1b shows the dependency of CaMAP activity on Ca²⁺/calmodulin concentration. With concentrations>5 mM of calcium chloride, precipitation of the sample was observed.

### EXAMPLE 2

### Screening by Means of Fluorescence Assay

To carry out the assay, an instruction described in patent specification WO0188178 and the appliance combination described in patent specification WO0102837 is used and modified as follows: the following solutions are prepared: substrate solution: 2 mg/ml disulfide bridged Abz-Cys-Phe-Pro-Ala-Cys-Phe-NHNp in DMSO; enzyme solution: 1 μM solution of human FKBP38; 5 μM calmodulin (preparation see Example 9), 5 mM CaCl₂ in 50 mM HEPES buffer, pH 7.5; effector solutions: 0.1 mg/ml substance in DMSO; starter solution: 100 mM DTT in 50 mM HEPES buffer, pH 7.5.

2 μl substrate solution, 1 μl effector solution and 20 μl enzyme solution are pipetted into each cell of a commercially available titer plate with 384 reaction cells. In order to reduce pipetting errors, a mixture was prepared of the respective amounts of enzyme and substrate solution so that, pipetting 40 μl of this mixture into each cell, the concentration is the same as when pipetting individual volumes. The plate then is stored at 6° C. for 20 minutes in such a way that, after 20 minutes, each of the reaction cells has a temperature of 6° C. The actual reaction is started by adding 20 μl starter solution to each cell.

Given appropriate constancy of temperature at 6° C. throughout the measuring time and achieving homogenous mixing of the solutions of substrate, enzyme and effector solutions with the starter solution, one can register in each single reaction cell the increase of visible light at 420 nm if fluorescence is induced by means of an ultraviolet lamp with an excitation spectrum range of between 250 and 330 nm. The visual comparison of the registration curves obtained can be used for the detection of an effector.

### EXAMPLE 3

### Screening by Means of Isomer-specific Hydrolysis

To carry out the assay, an instruction published in Clinical Chemistry 44(1998)502-508 is modified as follows: the following solutions are prepared: substrate solution: 30 mg/ml Suc-Ala-Phe-Pro-Phe-NHNp in DMSO; enzyme solution: 1 μM solution of human FKBP38; 5 μM calmodulin (Sigma: bovine heart calmodulin; purchase order no.: P0270), 5 mM CaCl₂ in 50 mM HEPES buffer, pH 7.5; effector solutions: 0.5 mg/ml substance in DMSO; starter solution: 20 mg/ml chymotrypsin in 50 mM HEPES buffer, pH 7.5.

20 μl substrate solution, 1 μl effector solution and 20 μl enzyme solution are pipetted into each cell of a commercially available titer plate With 96 reaction cells. In order to reduce pipetting errors, it is an advantage to prepare a mixture of the appropriate amounts of enzyme and substrate solution so that, pipetting 40 μl of this mixture into each cell, the concentration is the same as when pipetting individual volumes. The plate then is stored at 4° C. for 20 minutes in such a way that, after 20 minutes, each of the reaction cells has a temperature of 4° C. The actual reaction is started by adding 80 μl starter solution to each cell.

Given appropriate constancy of temperature at 6° C. throughout the measuring time and achieving homogenous mixing of the solutions of substrate, enzyme and effector solutions with the starter solution, the experimental setup published in Clinical Chemistry 44(1998)502-508 allows to obtain 96 registration curves within approximately 12 minutes. The visual comparison of the registration curves obtained can be used for the detection of an effector.

### EXAMPLE 4

### Searching for CaMAPs in Databases

Databases accessible to the person skilled in the art, such as Swiss-Prot; TrEMBL; Trenew; Trest; Trgen; Trome etc., which are accessible under http//www.expasy.com, allow relatively easy search for CaMAPs by entering calmodulin sequence motifs (cf.: FASEB J. 1997 April;11(5):331-40; Nature 410(2001)1120-1124). Searching with the helical CaM motif KHAAQRSTETALYRKM results in the following hits:
sw:AIP_CERAE, sw:AIP_HUMAN, sw:AIPL1_HUMAN, sw:AILP1_RAT, sw:AILP1_MOUSE, sw:AILP1_RABIT, sw:FKB5_HUMAN, sw:AIP_MOUSE, sw:CYP4_BOVIN, sw:FKB4_HUMAN, sw:FKBS5_MOUSE, sw:FKB4_MOUSE, sw:FKB4_RABIT, sw:CYP4_HUMAN, sw:FKB7_WHEAT, tr:Q9XT11, tr:Q9C650, tr:Q95L05, tr:Q9XSI2, tr:Q9LSF3, tr:Q38949, tr:Q07617, tr:Q904N1, tr:Q9VL78, tr:Q9LDC0, tr:O04843, tr:Q38931, tr:Q9XSH5, tr:Q9QZJ4, tn:AAM13008, tn:AAH15260, te:Hs_{—}75305_{—}4, te:Os_{—}10593_{—}1, te:BJ463801, te:BJ467593, te:BQ481189, te:BQ574171, te:W78674, te:At_{—}5664_{—}1, te:Dr_{—}5498_{—}2, te:Hs_{—}153057_{—}2, te:Hv_{—}2313_{—}1, te:Zm_{—}2379_{—}1, te:BJ468073, te:BJ463557, te:BJ466276, te:BQ458668, te:BQ425486, te:BG833626, te:Hs_{—}75305_{—}6, te:Hs_{—}153057_{—}3, te:Hv_{—}6100_{—}1, te:BI839989, te:BE222983, te:BG056407, te:At_{—}25402_{—}1, te:Bt_{—}4797_{—}1, te:Ta_{—}6047_{—}1, te:AV925548, te:BJ467976, te:BE455629, te:BQ287799, te:BQ205586, te:Mm_{—}154390_{—}2, te:Ta_{—}65_{—}1, te:Ta_{—}639_{—}1, te:BJ462559, te:BJ465793, te:AW43479, te:BG115973, te:BQ417256, te:At_{—}36868_{—}1, te:Bt_{—}7221_{—}1, te:Hs_{—}7557_{—}4, te:Ta_{—}639_{—}2, te:BJ463904, te:BF921901, te:Mm_{—}75161_{—}1, te:BQ238312, te:BJ467770, te:BJ485644, te:BJ468521, te:BI563211, te:BM034859, te:BQ421312, te:BQ575087, te:BQ574529, te:Dm_{—}1764_{—}1, te:Mm_{—}12758_{—}3, te:Rn_{—}8187_{—}2, te:Rn_{—}23741_{—}2, te:Zm_{—}3457_{—}1, te:BJ483495, te:BJ462661, te:BJ465277, te:BJ460115, te:BJ452568, te:BQ575306, tg:AC093196_{—}37, tg:AC020203_{—}5, tg:AC025647_{—}4, tg:AL590962_{—}2, tg:AC082643_{—}3, tg:AB026647_{—}15, tg:AB077822_{—}10, tg:AP001184_{—}15, tg:AB019232_{—}10, tg:AC005135_{—}13, tg:AP003474_{—}7, tg:AC005841_{—}4, tg:AE003626_{—}64, tg:AL033519_{—}4, tg:AL355494_{—}2, to:NT_{—}009759_{—}3_{—}6, to:NT_{—}007978_{—}123_{—}1, to:NT_{—}007978_{—}123_{—}2, to:NT_{—}009759_{—}3_{—}0, to:NT_{—}007592_{—}1412_{—}0, to:NT_{—}007592_{—}1412_{—}1, to:NT_{—}007592_{—}1412_{—}2, to:NT_{—}009759_{—}3_{—}4, to:NT_{—}007978_{—}123_{—}0.

Limiting the search algorithm to PPIases that are listed in the “Swiss-Prot” database, yields the following result for enzymes: AIP_HUMAN, AIP_CERAE, AIP_MOUSE, AIPL1_HUMAN, AILP1_RAT, AILP1_MOUSE, AILP1_RABIT, FKB8_HUMAN, FKB8_MOUSE, FKB5_HUMAN, FKB5_MOUSE, FKB4_HUMAN, FKB4_MOUSE, FKB4_RABIT, FKB7_WHEAT, CYP4_BOVIN, CYP4_HUMAN.

### EXAMPLE 5

### FKBP38 Inhibition by Means of FK506

The following solutions are prepared in order to detect the inhibition of the PPIase activity of CaMAP FKBP38: solution H: DMSO; solution I: 15 mM solution of FK506 (Fujisawa GmbH) in DMSO. According to Example 1, two cuvettes are charged with 1 μl solution B (substrate); 0.5 μM FKBP38, 0.5 μM calmodulin and 5 mM calcium chloride. Subsequently, 1 μl of solution H is added to cuvette 5a and, for comparison, 1 μl solution I is added to cuvette 5b. After mixing the solutions, the cuvettes are stored at 6° C. for 20 minutes. Subsequently, the determination of PPIase activity is started, as described in Example 1, adding solution C (chymotrypsin) by pipetting. When using substrate Suc-Ala-Phe-Pro-Phe-NHNp, the calculated evaluation of the isomerization velocity of the solution in cuvette 5a results in a value of 0.0071 s⁻¹ and in cuvette 5b in a value of 0.0105 s⁻¹. The value of 0.0071 corresponds to the uncatalyzed reaction in FIG. 1b. The value of 0.0105 corresponds to the catalyzed reaction of FIG. 1a. The progress curve 1d corresponds to the uncatalyzed reaction. Thus, the PPIase activity of CaMAP FKBP38 can be inhibited by the FKBP inhibitior FK506. Plotting the inhibitor concentration against the detected enzyme activity yields an IC50 value of about 4.3 μM.

### EXAMPLE 6

### Searching for Effectors by Means of Affinity Assay

Using this screening preparation, effected in titer plates (96 wells, flat bottom; Nunc Diagnostik), makes it possible to search for effectors that disturb the interaction between effective calmodulin and CaMAP that can be activated. This assay is carried out as radioactive assay. The following preparations have to be made:
a) Preparation of radioactively labeled calmodulin: Using human SrC-tyrosine kinase (Sigma: purchase order no.: 5439), calmodulin (recombinant human calmodulin produced according to Example 2) is specifically phosphorylated. To this aim, 10 μl tyrosine kinase, 50 μl calmodulin (1.2 mg/ml), 10 μl ³²P_{gamma} ATP (Amersham, newly delivered) amounting to a total volume of 80 μl are incubated at 30° C. for 3 hours, with the total preparation with a pH value of 7.5, in addition to calmodulin and tyrosine kinase, also containing 2 mM calcium chloride, 50 mM Tris buffer, 1 mM DTT, 100 mM NaCl, 10 mM MgCl₂, 1% glycerol and 0.01% Tween. The mixture is subsequently dissolved in 500 μl of a solution of 30% ammonium sulfate and 50 mM Tris buffer, pH 7.5 and applied to a chromatography column (Pierce, mini columns) filled with 2 ml phenyl sepharose (Sigma) and equilibrated with the application buffer. After the application, the column is washed with approximately 50 column volumes. Subsequently, the radioactively labeled calmodulin is eluted by gradually washing the column with a solution consisting of 50 mM Tris buffer/30% glycerine at a pH value of 7.5 in aliquots of 100 μl and separately collected in tubes. The fractions containing calmodulin can be detected due to their β-radiation by means of scintillation measuring and combined to a pooled fraction of usually 500 μl. Using not-radioactively labeled calmodulin and FKBP38 (production: see Example 9), a calmodulin/FKBP38 solution is prepared, which has a concentration of 5 μM calmodulin and 2.5 μM FKBP38.
b) Preparation of the antibody tagged titer plates: A solution of IgG-purified antibodies (obtained by immunisation of rabbits against HPLC-purified FKBP38) is diluted with 20 mM Tris buffer pH7.5 in 12 ten-fold dilution steps (1+9; 1+99; 1+999). The dilution is pipetted into the 12 rows of a 96-well titer plate in amounts of 50 μl in such a way that the first row contains the first dilution and the 12^{th} row contains the solution with the 12^{th} dilution. The titer plate is covered with a film and incubated at 4° C. for 12 hours. Subsequently, the portion of the antibody that is not bound is removed through carefully washing five times with wash solution (50 mM Tris buffer, pH 7.5; 0.1% Tween) and finally, the titer plate is supplied with 50 μl wash solution. Finally, the calmodulin/FKBP38 solution, obtained in a, is also diluted with working buffer (10 μM calcium chloride, 50 mM Tris buffer, 1 mM DTT, 100 mM NaCl, 1% glycerol and 0.01% Tween) in 8 ten-fold dilution steps. Then 1 μl of each of the dilutions are pipetted into the titer plate in such a way that row 1 contains the 1+9 dilution, row 2 the 1+99 dilution, etc. After incubating the titer plate at 4° C. for one hour, the titer plate is again washed five times with wash solution and, subsequently, put on a ³²P-sensitive screen (Amersham) for 1 hour and measured using phosphoimager (FUJI Diagnostik) and evaluated by means of the corresponding software. Optimum concentrations of CaMAP and antibody for carrying out the assay are assigned to the wells of the titer plate exhibiting high radioactivity. Highest assay sensitivity is observed near the transition from high to low radioactivity.
c) Carrying out the assay/quality assurance: A desired number of titer plates are prepared, as described in b), with the optimum concentrations of antibody and calmodulin established in b). Titer plates that are not correctly prepared can be detected using the phosphoimaging method mentioned. The actual screening is carried out as follows: 5 μl of the agent to be tested are pipetted into the 50 μl washing solution contained in each well. For quality assurance, 5 μl of 500 μM EDTA solution are pipetted into 2 of the wells. After incubating the titer plate at 4° C. for 1 hour, the plate is again washed five times with washing buffer in order to rinse off released radioactive calmodulin. The remaining radioactivity is subsequently measured using phosphoimaging. Effectors leading to the displacement of the activating calmodulin from the CaMAP, such as the unspecific EDTA, can be recognized by the reduced radioactivity in these wells.

### EXAMPLE 7

### Competition Assay with Radioactively Labeled Ligand

Competition assays for CaMAPs can be set up using already known inhibitors of CaMAPs, such as cyclosporin A or FK506. A simple solution consists of using radioactively labeled ligands which are available, for example, as tritium-labeled FK506 (Transplanation. 63(2):293-298, 1997; Fujisawa GmbH) or as Fujisawa tritium-labeled cyclosporin A (Amersham). In addition, a possible method that includes the binding of calmodulin to titer plates is described: this method takes advantage of the fact that streptavidin-coated microtiter plates (e.g.: Roche-Diagnostik; cat. no.: 1734776) and biotin-labelled calmodulin (Calbiochem; cat. no.: 208697) are commercially available as well as a standard microplate reader (Dynatech, MR7000) with an automatic dispensing unit and washer. In a first step, calmodulin (0.5 μM solution, 30 μl per well) is added to the coated titer plates, according to the manufacturer's instructions, at room temperature for 30 minutes. After rinsing five times with washing solution (see Example 6), the titer plate is ready for use and can be stored at room temperature for at least 8 hours. Just before starting the competition assay, 0.5 μM FKBP38 solution (50 mM Tris buffer, pH 7.5; 5 mM CaCl₂; 1 mM DTT; 0.5 mM glycerol) is added to the titer plate by pipetting 30 μl per well at room temperature for 30 minutes. Subsequently, the plate is washed again carefully five times. If the CaMAP used can be inhibited by cyclosporin A, the radioactive ligand subsequently used is tritium-labeled cyclosporin A; if the CaMAP, such as FKBP38, is inhibited by FK506, tritium-labeled FK506 is used as radioactive ligand. To carry out the assay, 0.5 μl agent (1 mg/ml in DMSO) and 30 μl buffer solution (50 mM Tris buffer, pH 7.5; 5 mM CaCl₂; 1 mM DTT; 0.5 mM glycerol) containing a 50 μM concentration of a radioactively labeled ligand are incubated at room temperature for 60 minutes. Subsequently, 20 μl are taken from each well, 1 ml scintillation solution (Roth) is added and measuring is carried out using Quickzint Flow Counter (Zinsser Analytic). Potential ligands are recognised by an increased radioactivity signal.

### EXAMPLE 8

### Inhibition of Cyp40 by Cyclosporin A

Cyp40 is prepared using a molecular biological method according to Acta Crystallographica Section D—Biological Crystallography. 55(Part 5):1079-1082, 1999.

The procedure is analogous to Example 3. 1 μM Cyp40 and 5 μM calmodulin plus 5 mM CaCl₂ dissolved in 50 mM HEPES buffer pH 7.5 are used as enzyme solution. As effector solution, 1 mg cyclosporin A (Sigma C3662) is dissolved in 50% ethanol. The control solution consists of a 50% ethanol solution.

The comparison of measuring with and without cyclosporin shows a reaction curve characteristic of inhibition.

### EXAMPLE 9

### Molecular Biological Method for the Production of CaMAP FKBP38

After identifying the potential CaMAP in accordance with Example 5, IRALp962N1726Q2 (RZPD-Ressourcenzentrum GmbH, Berlin) and the primers 38BspH15 and h383-ma were used to carry out a PCR preparation with 1.5 mM MgCl and 1 μl enhancer with Pfx-polymerase at an annealing temperature of 50° C., wherein the glycine in position 2 is substituted by an arginine. The PCR product was purified and cloned into the pSTBlue-1-vector by blunt end ligation. The product of the ligation was transformed into E. Coli DH5α cells. Positively selected transformants were transferred onto an agar plate with kanamycin and their cell material was used as template for a colony PCR. The PCR products of the positively selected clones were isolated and analytically digested with the restriction endonucleases NcoI and SacI. Subsequently, sequence hFKBP381-336 was cloned into pET28a. The resultant construct was transformed into Rosetta cells.

Six 1 l cultures of the clone were cultivated to produce approximately 100 mg FKBP38. Deviating from the standard conditions (Clontech/pET28a-vector), the cultures were cultivated at 20° C. Furthermore, 20 minutes before the induction by means of the addition of ethanol, a final concentration of 2% was established. After harvesting and disrupting the cell material using French® press and after subsequent ultra-centrifugation, 200 ml 10 mM MES buffer (pH 6.0) were added to the supernatant, which was subsequently subjected to various chromatographic purifying steps, such as ion exchange chromatography (DEAE, SO₃⁻) and gel permeation chromatography (Sephadex G75).

### EXAMPLE 10

### Radioactive Labeling of CaMAP FKBP38

10 μl of a FKBP38 solution (0.2 mg/ml, preparation according to Example 9) are incubated with 1 μl protein kinase A (NEB; purchase order no.: P6000L; protein amount 1 mg/ml) at a pH of 7.5 (50 mM Tris buffer) and 200 μM gamma³²P-ATP (Amersham, 8 Curie per mmol) at 30° C. for 1 hour. ³²P-labeled FKBP38 is purified from further radioactive components using gel filtration. FIG. 2 shows a comprehensive view, obtained via SDS-PAGE and phosphoimager, of the chromatographically pure CaMAP thus prepared.

### EXAMPLE 11

### Spectroscopically Detectable Structure Modifications

The CD spectra of FIG. 3a were taken at 20° C. with a CD spectrometer (Jasco J-710) and in a chambered 1 cm cuvette. The following conditions were chosen: solution A: 5 mM CaCl₂, 10 μM FKBP38 in CD buffer; solution B: 20 μM calmodulin (bovine brain, Sigma) in CD buffer. CD buffer refers to a solution consisting of 10 mM HEPES buffer and 5 mm calcium chloride with a pH of 7.5. The continuous line corresponds to the overall spectrum modification of solutions A and B in different chamber sections. The dotted line shows a characteristic spectrum which occurs after mixing both solutions. After adding 1 M EGTA to the mixture, the spectrum that is characteristic of the non-interacting proteins calmodulin and FKBP38 is obtained (continuous line).

The conditions resulting in FIG. 3b correspond to those of FIG. 3a: the dotted line shows the characteristic spectrum exhibited after mixing both solutions. The spectrum of the continuous line was obtained after increasing the calcium chloride concentration to 10 mM. This spectrum is characteristic of the precipitation of proteins.

1. Method for identifying and/or the producing an effector of a calmodulin-dependent peptidyl-prolyl cis/trans isomerase (CaMAP) consisting of the following steps:
(a) mixing of appropriate amounts of a CaMAP or a CaMAP peptide fragment/derivative with an appropriate amount of calmodulin or of a calmodulin fragment/derivative in an appropriate reaction solution with and without the effector;
(b) adding an appropriate amount of an appropriate CaMAP substrate,
(c) measuring CaMAP activity; and
(d) detecting that the effector is
(i) an inhibitor if the CaMAP activity in the reaction solution with the effector is lower than in the reaction solution without the effector; or
(ii) an activator if the CaMAP activity in the reaction solution with the effector is higher than in the reaction solution without the effector.

2. Method for screening and/or producing an effector of a CaMAP consisting of the steps of
(a) mixing appropriate amounts of a CaMAP or a CaMAP peptide fragment/derivative with an appropriate amount of calmodulin or a calmodulin fragment/derivative in an appropriate reaction solution with and without a sample containing a single or a multitude of compounds which are candidates for an inhibitor or an activator;
(b) adding an appropriate amount of an appropriate CaMAP substrate;
(c) measuring CaMAP activity; and
(d) detecting that the sample
(i) exhibits inhibitory activity if the CaMAP activity in the reaction solution with the sample is lower than in the reaction solution without the sample; or
(ii) exhibits activating activity if the CaMAP activity in the reaction solution with the sample is higher than in the solution without the sample.

3. Method according to claim 2, further comprising step
(e) fractioning of the sample for which inhibitory or activating activity was detected in step (d) and repeating of steps (a) to (d) until the inhibitor or activator contained in the sample is present in purified form.

4. Method according to claim 1, wherein the CaMAP is selected from the group consisting of the human CaMAPs FKBP36, FKBP37.7, FKBP44, FKBP51, FKBP52 and Cyp40 and enzymes that are listed in the “Swiss-Prot” database corresponding to the denotation used in this database under FKBP66, FKBP42, AIP_HUMAN, AIP_CERAE, AIP_MOUSE, AIPL1_HUMAN, AILP1_RAT, AILP1_MOUSE, AILP1_RABIT, FKB8_HUMAN, FKB8_MOUSE, FKB5_HUMAN, FKB5_MOUSE, FKB4_HUMAN, FKB4_MOUSE, FKB4_RABIT, FKB7_WHEAT,
CYP4_BOVIN and CYP4_HUMAN.

5. Method according to claim 1, wherein the calmodulin or the calmodulin fragment/derivative is selected from the group consisting of
CALM_ACHKL (P15094), CALM_BLAEM (Q9HFY6), CALM_CANAL (P23286), CALM_CAPAN (P93087), CALM_CHLRE (P04352), CALM_DICDI (P02599), CALM_DROME (P07181), CALM_ELEEL (P02594), CALM_EMENI (P19533), CALM_EUGGR (P11118), CALM_FAGSY (Q39752), CALM_HELAN (P93171), CALM_HORVU (P13565), CALM_HUMAN (P02593), CALM_KLULA (O60041), CALM_LYCES (P27161), CALM_LYTPI (P05935), CALM_MAGGR (Q9UWF0), CALM_MAIZE (P41040), CALM_MALDO (P48976), CALM_MEDSA (P17928), CALM_METSE (P02596), CALM_NEUCR (Q02052), CALM_ORYSA (P29612), CALM_PARTE (P07463), CALM_PATSP (P02595), CALM_PHYIN (P27165), CALM_PLAFA (P24044), CALM_PLECO (P11120), CALM_PNECA (P41041), CALM_PYUSP (P11121), CALM_SCHPO (P05933), CALM_SOLTU (P13868), CALM_SPIOL (P04353), CALM_STIJA (P21251), CALM_STRPU (P05934), CALM_STYLE (P27166), CALM_TETPY (P02598), CALM_TETTH (Q05055), CALM_TRYBB (P04465), CALM_TRYCR (P18061), CALM_WHEAT (P04464), CALM_YEAST (P06787), Q9UWF0, Q02052, P19533, AAL89686, Q7M510, Q96TN0, P27165, AAG01043, P02593, Q7T3T2, Q40302, O02367, Q95NR9, Q9UB37, AAR54805 AAH54973, AAL02363, AAH59427, AAH59500, AAH54600, AAH53150, AAH50926, AAH45298, AAH44434, AAP88918, AAP35501, AAP35464, BAC56543, AAC83174, AAD55398, AAC63306, AAD45181, AAH21347, BAC40168, BAB28631, BAB28319, BAB28116, BAB23462, AAH58485, AAH51444, AAH47523, P07181, Q7QGY7, Q8STF0, AAO25039, AAM50750, AAK61380, BAB89360, O94739, P02594, Q9D6G4, O16305, Q96HK3, P11120, O96102, P21251, Q9U6D3, Q8X187, O93410, AAR10240, P11121, Q9XZP2, Q42478, AAQ01510, P17928, P93171, O97341, O96081, AAD10244, AAM81203, AAA34238, AAA34014, AAA34013, P02596, P93087, Q43699, CAD20351, BAB61916, BAB61915, AAF65511, P02595, P59220, P27162, Q93VL8, Q39447, Q94801, AAQ63462, AAQ63461, AAM81202, BAB61918, BAB61917, BAB61914, BAB61913, BAB61912, BAB61911, BAB61910, BAB61909, AAG27432, AAG11418.

6. Method according to claim 1, wherein the appropriate reaction solution contains bivalent ions selected from the group consisting of Zn²⁺, Cu²⁺, Co²⁺, Ni²⁺, Mn²⁺, Ca²⁺ and/or Mg²⁺ at a concentration of 0.1 to 20 mM.

7. Method according to claim 1, wherein the appropriate reaction solution has a pH of between pH 5 and pH 10.

8. Method for identifying and/or producing an effector of a CaMAP consisting of the steps
(a) mixing appropriate amounts of a constitutively active CaMAP in an appropriate reaction solution with and without effector;
(b) adding an appropriate amount of an appropriate CaMAP substrate;
(c) measuring CaMAP activity; and
(d) detecting that the effector is
(i) an inhibitor if the CaMAP activity in the reaction solution with the effector is lower than in the reaction solution without the effector; or
(ii) an activator if the CaMAP activity in the reaction solution with the effector is higher than in the reaction solution without the effector.

9. Method according to claim 1, wherein steps (a) and (b) are interchanged.

10. Method according to claim 1, wherein the detection is carried out by spectroscopic or radioactive methods.

11. Method according to claim 1, wherein the method is a high-throughput method.

12. Method according to claim 1, further comprising step
(f) formulating the identified and/or produced effector with a pharmaceutically acceptable carrier or solvent.

13. Compound identified according to the method of claim 1, wherein the effector is a cycloheximide derivative having the general formula (1): 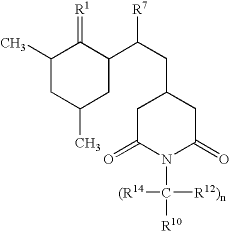
in which n is an integer from 1 to 20; R¹² independently is a hydrogen atom, an alkyl residue or an aryl residue,
R¹ is selected from an oxygen atom, a sulfur atom, or the groups NR², NOR² and N—NR²R³, wherein
(a) R² and R³, independently from each other, are a hydrogen atom, aryl or alkyl, respectively, which can optionally be interrupted by O, S, NH, NR⁵, aryl, heteroaryl, cycloalkyl, heterocycloalkyl or can optionally be substituted by R⁶, or
(b) R² and R³, together, are C₁-C₆-alkylene, which can optionally be interrupted by O, S, NH, NR⁵, aryl, heteroaryl, cycloalkyl or heterocycloalkyl or can optionally be substituted by R⁶, wherein
R⁵ is an alkyl residue or an aryl residue,
R⁶ stands for a hydrogen atom, alkyl, aryl, OR⁵, C(O)OR⁵, CN, F or Cl, wherein R⁵ is defined as above,
R⁷ is a —OH, —OR⁹, —OC(O)R⁹, —OC(S)R⁹, —OC(O)NHR⁹ or —OC(S)NHR⁹ residue, wherein
R⁹ is an alkyl residue which can optionally be interrupted by O, S, NH, NR⁵, aryl, heteroaryl, cycloalkyl or heterocycloalkyl or can optionally be substituted by R⁶ as defined above, or alternatively
R⁹ is an aryl residue which can optionally be interrupted by O, S, NH or NR⁵ or can optionally be substituted by R⁶ as defined above,
R¹⁰ is a —NHR², —NR²R³, —C(O)OR², —C(S)OR², —C(O)NR²R³, —CN, —NR²C(O)NR²R³, —OC(O)NR²R³, —NR²C(S)NR²R³, —OC(S)NR²R³, or OR², C(O)NHR¹¹ residue, wherein R² and R³ are defined as above,
R¹¹ stands for an amino acid residue or an oligopeptide residue and
R¹⁴ is an alkyl residue or an aryl residue.

14. Compound identified according to the method of claim 1, wherein the effector is a cyclohexamide derivative having the general formula (1) in which n is an integer of 1 to 20 and exhibiting an ether group between R¹⁵ and the complete molecule as illustrated in formula (2) below, 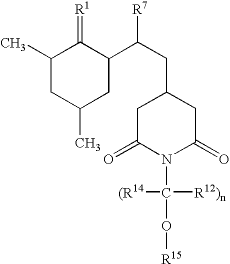
R¹ is selected from an oxygen atom, a sulfur atom, or the groups NR², NOR² and N—NR²R³, wherein
(a) R² and R³, independently from each other, are a hydrogen atom, aryl or alkyl, respectively, which can optionally be interrupted by O, S, NH, NR⁵, aryl, heteroaryl, cycloalkyl, heterocycloalkyl or can optionally be substituted by R⁶, or
(b) R² and R³, together, are C₁-C₆-alkylene, which is optionally interrupted by O, S, NH, NR⁵, aryl, heteroaryl, cycloalkyl or heterocycloalkyl or can optionally be substituted by R⁶, wherein
R⁵ is an alkyl residue or an aryl residue,
R⁶ stands for a hydrogen atom, alkyl, aryl, OR⁵, C(O)OR⁵, CN, F or Cl, wherein R⁵ is defined as above,
R⁷ is a —OH, —OR⁹, —OC(O)R⁹, —OC(S)R⁹, —OC(O)NHR⁹ or —OC(S)NHR⁹ residue, wherein
R⁹ is an alkyl residue which can optionally be interrupted by O, S, NH, NR⁵, aryl, heteroaryl, cycloalkyl or heterocycloalkyl or can optionally be substituted by R⁶ as defined above, or alternatively
R⁹ is an aryl residue which can optionally be interrupted by O, S, NH or NR⁵ or can optionally be substituted by R⁶ as defined above,
R¹¹ stands for an amino acid residue or an oligopeptide residue,
R¹⁴ is a hydrogen atom, and
R¹⁵ is a hydrogen atom, an alkyl or an aryl residue.

15. Compound according to claim 13 having the above-identified formula (1) for which the following applies:
(a) n=1, 2, 3; R¹=O; R⁷=OH, O(CHR¹²)ₙR¹⁰, OC(O)CH₃; R¹⁰=C(O)OCH₃, C(O)OC₂H₅, CN, C(O)NH₂,
(b) n=3-10; R¹=O; R⁷=OH; R¹⁰=C(O)NHR¹¹, R¹¹=amino acid residue, oligopeptide residue,
(c) n=1, 2, 3; R¹=O; R⁷=OH, O(CHR¹²)ₙR¹⁰; R¹⁰=C(O)OCH₃, C(O)OC₂H₅, CN, C(O)NH₂,
(d) n=1, 2, 3; R¹=NOH, N—NHPh, N—NHCH₃, N-alkyl, N-benzyl; R⁷=OH, O(CHR¹²)ₙR¹⁰; R¹⁰=C(O)OCH₃, C(O)OC₂H₅, CN, C(O)NH₂,
(e) n=1, 2, 3; R¹=O; R⁷=OH, O(CHR¹²)ₙR¹⁰, OC(O)NH-alkyl, OC(O)NH-cycloalkyl, OC(O)NH-aryl; R¹⁰=C(O)OCH₃, C(O)OC₂H₅, CN, C(O)NH₂.

16. Compound according to claim 13 selected from one of the following compounds:

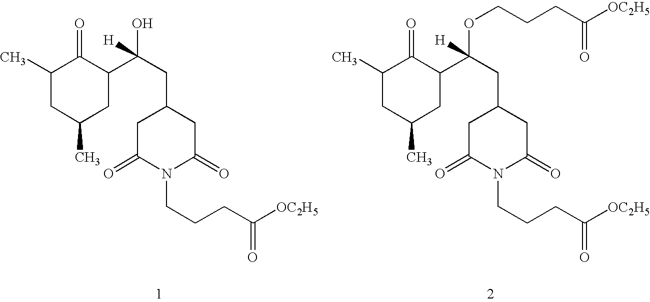

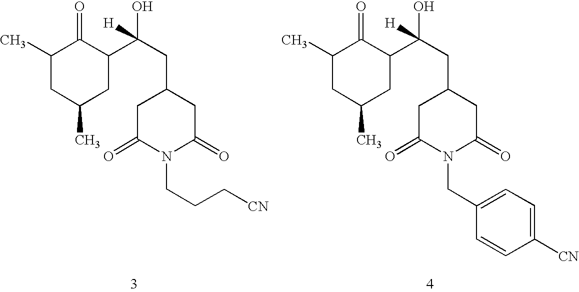

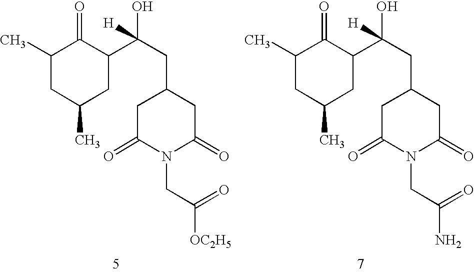

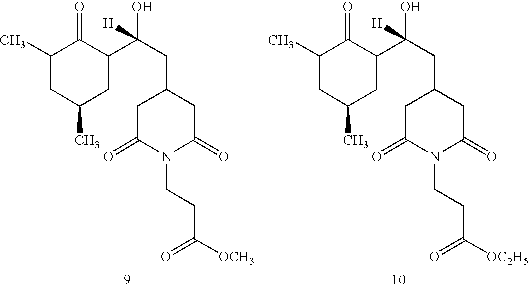

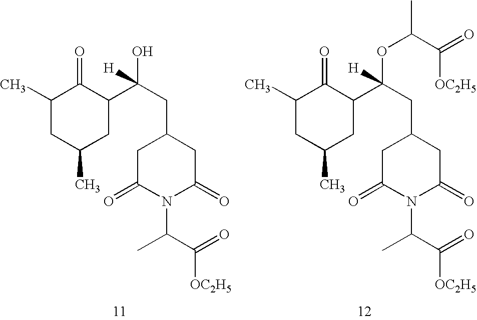

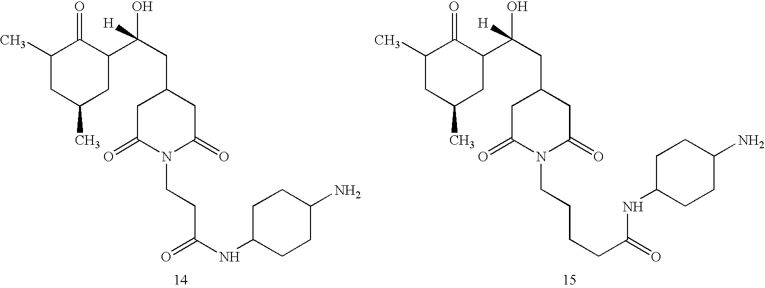

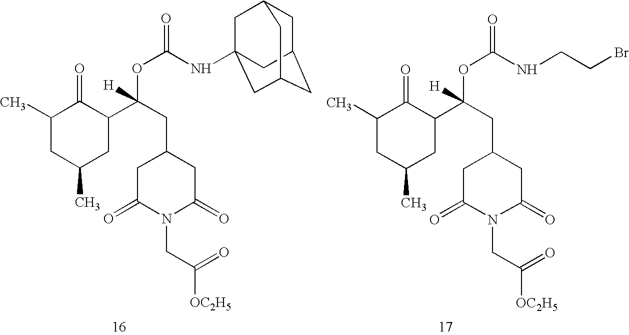

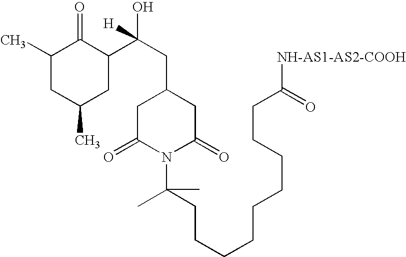

| compound | amino acid residue AS1 | amino acid residue AS2 |
|---|---|---|
| 18 | alanine | alanine |
| 19 | valine | alanine |
| 20 | tryptophan | alanine |
| 21 | isoleucine | alanine |
| 22 | methionine | alanine |
| 23 | glycine | alanine |
| 24 | alanine | valine |
| 25 | valine | valine |
| 26 | tryptophan | valine |
| 27 | isoleucine | valine |
| 28 | methionine | valine |
| 29 | glycine | valine |

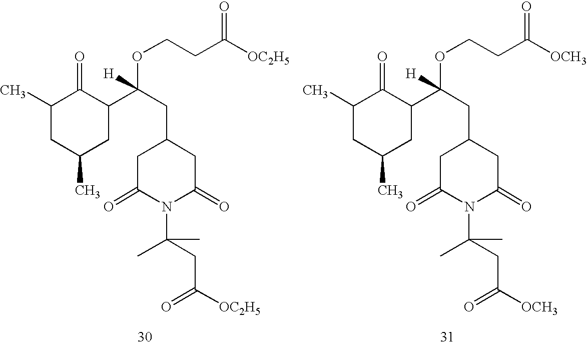

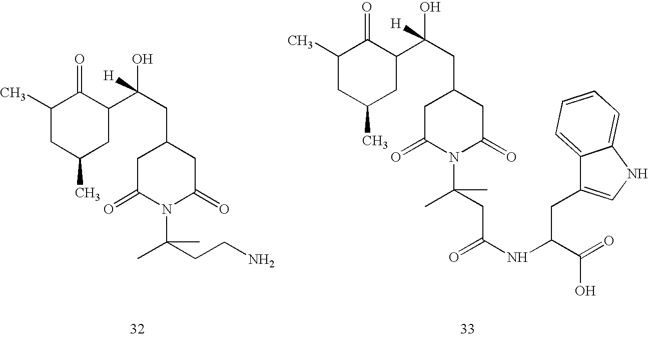

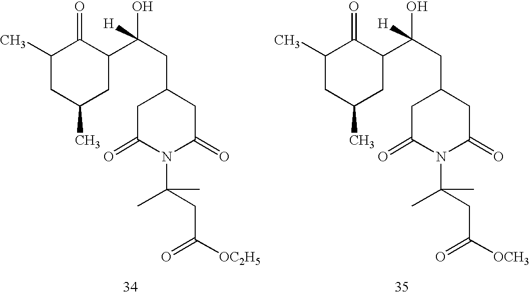

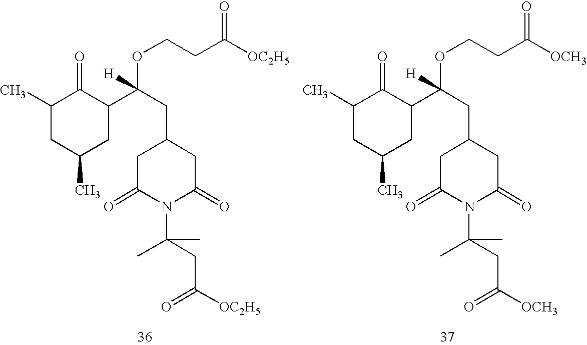

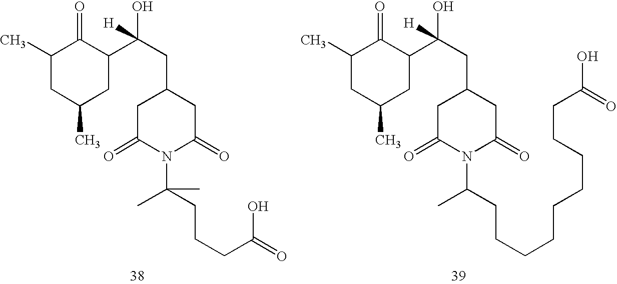

17. The effector identified and/or produced by the process of claim 1, optionally with a pharmaceutically acceptable carrier or solvent.

18. A method for the treatment of tumour diseases comprising administering an effector of claim 1 to a patient in need thereof in an amount effective to treat a tumour disease.

19. A method for the inhibition or attenuation of transplant rejection or for the treatment of neurodegenerative diseases comprising administering an effector of claim 1 to a patient in need thereof in an amount effective to inhibit or attenuate transplant rejection or to treat neurodegenerative disease.

20. A kit comprising CaMAP or a peptide fragment/derivative as described in claim 1 and calmodulin or a calmodulin fragment/derivative as described in claim 1, one or more buffer solutions and/or one or more substrates.
